(19) United States
(12) Patent Application Publication
(10) Pub. No.: US 2022/0033389 A1
(43) Pub. Date: Feb. 3, 2022

(54) INSECTICIDAL COMPOUNDS

(71) Applicant: SYNGENTA CROP PROTECTION AG, Basel (CH)

(72) Inventors: Aurelien BIGOT, Stein (CH); André JEANGUENAT, Stein (CH); Myriem EL QACEMI, Stein (CH)

(73) Assignee: SYNGENTA CROP PROTECTION AG, Basel (CH)

(21) Appl. No.: 17/278,922

(22) PCT Filed: Sep. 20, 2019
(86) PCT No.: PCT/EP2019/075394

§ 371 Date: Mar. 23, 2021

(30) Foreign Application Priority Data
Sep. 26, 2018 (EP) 18196932.0

(51) Int. Cl. C07D 413/12; A01N 43/80

(52) U.S. Cl. CPC C07D 413/12; A01N 43/80

(57) ABSTRACT

Compounds of formula (I), wherein the substituents are as defined in claim 1, and the agrochemically acceptable salts, stereoisomers, enantiomers, tautomers and N-oxides of those compounds, and their uses as insecticides.

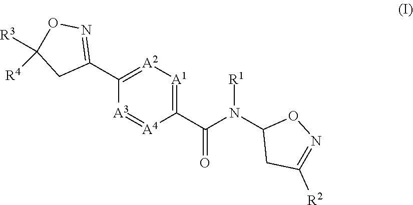

The present invention relates to certain isoxazoline derivatives, to intermediates for preparing such derivatives, to insecticidal, acaricidal, nematicidal and molluscicidal compositions comprising such derivatives and to methods of using these derivatives to control insect, acarine, nematode and mollusc pests.

Certain isoxazoline derivatives with insecticidal properties are disclosed, for example, in WO2011067272. It has now surprisingly been found that certain isoxazoline derivatives have highly potent insecticidal properties.

Hence, in a first aspect, as embodiment 1, there is provided a compound of formula (I),

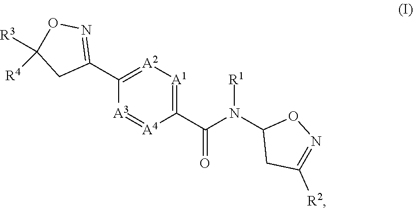

wherein

A¹, A², A³ and A⁴ are, independently of one another, C—H, C—R⁵ or N;

R¹ is selected from —(C₀-C₄alkyl)-C(═O)—C₃-C₆cycloalkyl, —(C₀-C₄alkyl)-O—C(═O)—C₃-C₆cycloalkyl, —(C₀-C₄alkyl)-O—C₃-C₆cycloalkyl, —R^{1a}OR^{1a}OR^{1b}, —R^{1a}OC(═O)R^{1b}, —R^{1a}OC(═O)OR^{1b}, —R^{1a}N(R^{1c})C(═O)OR^{1b}, —R^{1a}OC(═O)N(R^{1b})(R^{1c}), —R^{1a}C(═O)N(R^{1b})(R^{1c}) and —S—C(═O)OR^{1b};

R^{1a} is —(CR^{1d}R^{1e})ₙ—;

R^{1b} and R^{1c} are independently selected from H and C₁-C₄alkyl, wherein each alkyl group is unsubstituted or substituted with one to three halogen atoms or with a cyano group;

R^{1d} and R^{1e} are independently selected from H and C₁-C₄alkyl;

n is selected from 1,2,3 and 4;

R² is hydrogen, halogen, cyano, C₁-C₈alkyl or C₁-C₈alkyl substituted by one to three R^{6a}, C₁-C₈haloalkyl or C₁-C₈haloalkyl substituted by one to three R^{6a}, C₁-C₈alkoxy or C₁-C₈alkoxy substituted by one to three R^{6a}, C₃-C₈cycloalkyl or C₃-C₈cycloalkyl substituted by one to three R^{6b}, C₃-C₈cycloalkyl where one carbon atom is replaced by O, S, S(O) or SO₂, or C₃-C₈cycloalkyl-C₁-C₈alkyl, C₃-C₈cycloalkyl-C₁-C₈alkyl where one carbon atom in the cycloalkyl group is replaced by O, S, S(O) or SO₂, or C₃-C₈cycloalkyl-C₁-C₈haloalkyl, C₂-C₈alkenyl or C₂-C₈alkenyl substituted by one to three R^{6a}, C₂-C₈haloalkenyl or C₂-C₈haloalkenyl substituted by one to three R^{6a}, C₂-C₈alkynyl, C₂-C₈haloalkynyl, phenyl, phenyl substituted by one to three R⁷, phenyl-C₁-C₄alkyl, phenyl-C₁-C₄alkyl wherein the phenyl moiety is substituted by one to three R⁷, 5-6 membered heteroaryl, 5-6 membered heteroaryl substituted by one to three R⁷, 5-6 membered heteroaryl-C₁-C₄alkyl or 5-6 membered heteroaryl-C₁-C₄alkyl wherein the heteroaryl moiety is substituted by one to three R⁷, —NH(R⁸), —N(R⁸)(R⁹), —OR¹⁰, —SR¹⁰, —S(O)R¹⁰, —S(O)₂R¹⁰, COR¹⁰, COOR¹⁰;

R³ is C₁-C₈haloalkyl;

R⁴ is phenyl or phenyl substituted by one to three R^{6b} or pyridine or pyridine substituted by one to three R^{6b};

R⁵ is independently halogen, cyano, nitro, C₁-C₈alkyl, C₃-C₈cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy, or C₁-C₈alkoxycarbonyl-, or two R⁵ on adjacent carbon atoms together form a —CH═CH—CH═CH— bridge or a —N═CH—CH═CH— bridge;

each R^{6a} is independently halogen, cyano, nitro, amino, hydroxy, oxo, C₁-C₈alkylamino, hydroxyimino, C₁-C₈alkyloxyimino, di-C₁-C₈alkylamino, C₁-C₈alkoxy, acetyloxy, formyloxy, C₁-C₈haloalkoxy, C₁-C₄alkylthio or tri-(C₁-C₄alkyl)silyl;
each R^{6b} is independently halogen, cyano, nitro, C₁-C₈alkyl, C₁-C₈haloalkyl, amino, C₁-C₈alkylamino, di-C₁-C₈alkylamino, hydroxyl, C₁-C₄alkylthio, C₁-C₈alkoxy or C₁-C₈haloalkoxy; R⁷ is independently halogen, cyano, nitro, C₁-C₈alkyl, C₁-C₈haloalkyl, C₁-C₈alkoxy, or C₁-C₈haloalkoxy;

R⁸ and R⁹ are independently hydrogen, cyano, C₁-C₈alkyl or C₁-C₈alkyl substituted by one to three R^{6a}, C₁-C₈alkoxy, C₁-C₈haloalkoxy, C₁-C₈haloalkoxy substituted by one to three R^{6a}, C₁-C₈alkoxy substituted by one to three R^{6a}, C₁-C₈haloalkyl or C₁-C₈haloalkyl substituted by one to three R^{6a}, C₃-C₈cycloalkyl or C₃-C₈cycloalkyl substituted by one to three R^{6b}, C₃-C₈cycloalkyl where one carbon atom is replaced by O, S, S(O) or SO₂, or C₃-C₈cycloalkyl-C₁-C₈alkyl, C₃-C₈cycloalkyl-C₁-C₈alkyl where one carbon atom in the cycloalkyl group is replaced by O, S, S(O) or SO₂, or C₃-C₈cycloalkyl-C₁-C₈haloalkyl, C₂-C₈alkenyl or C₂-C₈alkenyl substituted by one to three R^{6a}, C₂-C₈haloalkenyl or C₂-C₈haloalkenyl substituted by one to three R^{6a}, C₂-C₈alkynyl, C₂-C₈haloalkynyl, phenyl, phenyl substituted by one to three R⁷, phenyl-C₁-C₄alkyl, phenyl-C₁-C₄alkyl wherein the phenyl moiety is substituted by one to three R⁷, 5-6 membered heteroaryl, 5-6 membered heteroaryl substituted by one to three R⁷, 5-6 membered heteroaryl-C₁-C₄alkyl or 5-6 membered heteroaryl-C₁-C₄alkyl wherein the heteroaryl moiety is substituted by one to three R⁷, or R⁸ and R⁹ together with the nitrogen atom can be linked through a C₃-C₈alkylene chain, a C₃-C₈alkylene chain substituted by one to three R^{6b} or a C₃-C₈alkylene chain, where one carbon atom is replaced by O, S, S(O) or SO₂;

R¹⁰ is hydrogen, cyano, C₁-C₈alkyl or C₁-C₈alkyl substituted by one to three R^{6a}, C₁-C₈haloalkyl or C₁-C₈haloalkyl substituted by one to three R^{6a}, C₃-C₈cycloalkyl or C₃-C₈cycloalkyl substituted by one to three R^{6b}, C₃-C₈cycloalkyl where one carbon atom is replaced by O, S, S(O) or SO₂, or C₃-C₈cycloalkyl-C₁-C₈alkyl, C₃-C₈cycloalkyl-C₁-C₈alkyl where one carbon atom in the cycloalkyl group is replaced by O, S, S(O) or SO₂, or C₃-C₈cycloalkyl-C₁-C₈haloalkyl, C₂-C₈alkenyl or C₂-C₈alkenyl substituted by one to three R^{6a}, C₂-C₈haloalkenyl or C₂-C₈haloalkenyl substituted by one to three R^{6a}, C₂-C₈alkynyl, C₂-C₈haloalkynyl, phenyl, phenyl substituted by one to three R⁷, phenyl-C₁-C₄alkyl, phenyl-C₁-C₄alkyl wherein the phenyl moiety is substituted by one to three R⁷, 5-6 membered heteroaryl, 5-6 membered heteroaryl substituted by one to three R⁷, 5-6 membered heteroaryl-C₁-C₄alkyl or 5-6 membered heteroaryl-C₁-C₄alkyl wherein the heteroaryl moiety is substituted by one to three R⁷;

or an agrochemically acceptable salt, tautomer or N-oxide thereof.

Compounds of formula (I) which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula (I) which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

The alkyl groups occurring in the definitions of the substituents can be straight-chain or branched and are, for example, methyl, ethyl, n-propyl, isopropyl, n-butyl, sec-butyl, iso-butyl, tert-butyl, pentyl, hexyl, nonyl, decyl and their branched isomers. Alkylsulfanyl, alkylsulfinyl, alkylsulfonyl, alkoxy, alkenyl and alkynyl radicals are derived from the alkyl radicals mentioned. The alkenyl and alkynyl groups can be mono- or polyunsaturated.

Halogen is generally fluorine, chlorine, bromine or iodine. This also applies, correspondingly, to halogen in combination with other meanings, such as haloalkyl or halophenyl.

Haloalkyl groups preferably have a chain length of from 1 to 6 carbon atoms. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl.

Alkoxy is, for example, methoxy, ethoxy, propoxy, i-propoxy, n-butoxy, isobutoxy, sec-butoxy and tert-butoxy and also the isomeric pentyloxy and hexyloxy radicals.

Alkoxyalkyl groups preferably have a chain length of 1 to 6 carbon atoms.

Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

Alkoxycarbonyl is for example methoxycarbonyl (which is C₁alkoxycarbonyl), ethoxycarbonyl, propoxycarbonyl, isopropoxycarbonyl, n-butoxycarbonyl, tert-butoxycarbonyl, n-pentoxycarbonyl or hexyloxycarbonyl.

The cycloalkyl groups preferably have from 3 to 6 ring carbon atoms, for example cyclopropyl, cyclobutyl, cyclopentyl and cyclohexyl.

Heteroaryl groups preferably are the 5-6 membered heteroaryls or the 5-6 membered heteroaryls substituted by one to three R⁷ where the heteroaryl groups contain 1 to 3 hetero atoms selected from the group consisting of nitrogen, oxygen and sulfur, it not being possible for each ring system to contain more than 2 oxygen atoms and more than 2 sulfur atoms. Examples of monocyclic groups include pyridyl, pyridazinyl, pyrimidinyl, pyrazinyl, pyrrolyl, pyrazolyl, imidazolyl, triazolyl (e.g. 1.2.4 triazoyl), furanyl, thiophenyl, oxazolyl, isoxazolyl, oxadiazolyl, thiazolyl, isothiazolyl, tetrazolyl and thiadiazolyl.

The compounds of formula (I) according to the invention also include hydrates which may be formed during the salt formation.

Preferred values of A¹, A², A³, A⁴, R¹, R², R³, R⁴, R⁵, R^{6a}, R^{6b}, R⁷, R⁸, R⁹ and R¹⁰ in relation to each compound of the present invention, including the intermediate compounds, are, in any combination (including combinations of preferred values with the original values) as set out below in the embodiments.

Embodiment 2: The compounds according to embodiment 1, wherein A¹ is C—R⁵; A² is C—H; A³ is C—H; and A⁴ is C—H, wherein R⁵ is halogen, cyano, nitro, C₁-C₈alkyl, C₃-C₈cycloalkyl, C₁-C₈haloalkyl, or C₂-C₈alkenyl.

Embodiment 3: The compound according to embodiment 1 or 2 wherein R² is selected from halogen, C₁-C₄alkyl, C₃-C₈cycloalkyl, C₁-C₄haloalkyl, C₁-C₄alkoxy, C₁-C₄haloalkoxy, di-C₁-C₄alkylamino, C₁-C₄alkylthio and C₁-C₄alkyloxycarbonyl.

Embodiment 4: The compound according to any one of embodiments 1 to 3 wherein R² is C₁-C₄alkoxy.

Embodiment 5: The compound according to any one of embodiments 1 to 4 wherein R¹ is selected from —(C₀-C₄alkyl)-C(═O)—C₃-C₆cycloalkyl, —(C₀-C₄alkyl)-O—C(═O)—C₃-C₆cycloalkyl, —R^{1a}OC(═O)R^{1b}, —R^{1a}OC(═O)OR^{1b}, —R^{1a}N(R^{1c})C(═O)OR^{1b} and —R^{1a}OC(═O)N(R^{1b})(R^{1c}).

Embodiment 6: The compound according to any one of embodiments 1 to 5 wherein R³ is C₁-C₄haloalkyl.

Embodiment 7: The compound according to any one of embodiments 1 to 6 wherein R⁴ is phenyl or phenyl substituted by one to three R^{6b}

R^{6b} independently is halogen, cyano, nitro, C₁-C₄alkyl, C₁-C₄haloalkyl, C₁-C₄alkoxy, or C₁-C₄haloalkoxy; more preferably bromo, chloro, fluoro, cyano, nitro, methyl, ethyl, trifluoromethyl, methoxy, difluoromethoxy and trifluoromethoxy.

Embodiment 8: The compound according to embodiment 1 represented by the compounds of formula (Ia)

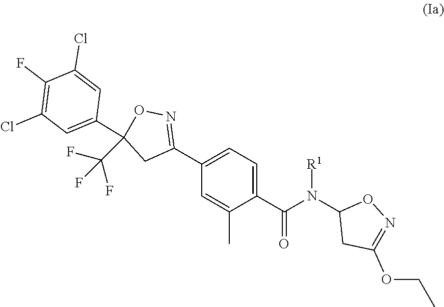

wherein

R¹ is selected from —(C₀-C₄alkyl)-C(═O)—C₃-C₆cycloalkyl, —(C₀-C₄alkyl)-O—C(═O)—C₃-C₆cycloalkyl, —(C₀-C₄alkyl)-O—C₃-C₆cycloalkyl, —R^{1a}OR^{1a}OR^{1b}, —R^{1a}OC(═O)R^{1b}, —R^{1a}OC(═O)OR^{1b}, —R^{1a}N(R^{1c})C(═O)OR^{1b}, —R^{1a}OC(═O)N(R^{1b})(R^{1c}), —R^{1a}C(═O)N(R^{1b})(R^{1c}) and —S—C(═O)OR^{1b};

R^{1a} is —(CR^{1d}R^{1e})ₙ—;

R^{1b} and R^{1c} are independently selected from H and C₁-C₄alkyl, wherein each alkyl group is unsubstituted or substituted with one to three halogen atoms or with a cyano group;

R^{1d} and R^{1e} are independently selected from H and C₁-C₄alkyl;

n is selected from 1,2,3 and 4.

Embodiment 9: The compounds according to embodiment 8, wherein R¹ is selected from —(C₀-C₄alkyl)-C(═O)—C₃-C₆cycloalkyl, —(C₀-C₄alkyl)-O—C(═O)—C₃-C₆cycloalkyl, —R^{1a}OC(═O)R^{1b}, —R^{1a}OC(═O)OR^{1b}, —R^{1a}N(R^{1c})C(═O)OR^{1b} and —R^{1a}OC(═O)N(R^{1b})(R^{1c});

R^{1a} is —(CR^{1d}R^{1e})ₙ—;

R^{1b} and R^{1c} are independently selected from H and C₁-C₄alkyl, wherein each alkyl group is unsubstituted or substituted with one to three halogen atoms or with a cyano group;

R^{1d} and R^{1e} are independently selected from H and C₁-C₄alkyl;

n is selected from 1,2,3 and 4.

Embodiment 10: The compounds according to any one of embodiments 1 to 9 wherein the compounds are selected from

| | |
|---|---|
| A1 | N-(cyclopropanecarbonyl)-4-[(5S)-5-(3,5-dichloro-4-fluoro-phenyl)-5-(trifluoromethyl)-4H- |
| | isoxazol-3-yl]-N-(3-ethoxy-4,5-dihydroisoxazol-5-yl)-2-methyl-benzamide |
| A2 | [[4-[(5S)-5-(3,5-dichloro-4-fluoro-phenyl)-5-(trifluoromethyl)-4H-isoxazol-3-yl]-2-methyl- |
| | benzoyl]-(3-ethoxy-4,5-dihydroisoxazol-5-yl)amino]methyl 2-methylpropanoate |
| A3 | [[4-[(5S)-5-(3,5-dichloro-4-fluoro-phenyl)-5-(trifluoromethyl)-4H-isoxazol-3-yl]-2-methyl- |
| | benzoyl]-(3-ethoxy-4,5-dihydroisoxazol-5-yl)amino]methyl 2,2-dimethylpropanoate |
| A4 | methyl N-[[[4-[(5S)-5-(3,5-dichloro-4-fluoro-phenyl)-5-(trifluoromethyl)-4H-isoxazol-3-yl]-2- |
| | methyl-benzoyl]-(3-ethoxy-4,5-dihydroisoxazol-5-yl)amino]methyl]-N-methyl-carbamate |
| A5 | [[4-[(5S)-5-(3,5-dichloro-4-fluoro-phenyl)-5-(trifluoromethyl)-4H-isoxazol-3-yl]-2-methyl- |
| | benzoyl]-(3-ethoxy-4,5-dihydroisoxazol-5-yl)amino]methyl N,N-dimethylcarbamate |
| A6 | [[4-[(5S)-5-(3,5-dichloro-4-fluoro-phenyl)-5-(trifluoromethyl)-4H-isoxazol-3-yl]-2-methyl- |
| | benzoyl]-(3-ethoxy-4,5-dihydroisoxazol-5-yl)amino]methyl methyl carbonate |
| A7 | [[4-[(5S)-5-(3,5-dichloro-4-fluoro-phenyl)-5-(trifluoromethyl)-4H-isoxazol-3-yl]-2-methyl- |
| | benzoyl]-(3-ethoxy-4,5-dihydroisoxazol-5-yl)amino]methyl cyclopropanecarboxylate |
| A8 | [[4-[(5S)-5-(3,5-dichloro-4-fluoro-phenyl)-5-(trifluoromethyl)-4H-isoxazol-3-yl]-2-methyl- |
| | benzoyl]-(3-ethoxy-4,5-dihydroisoxazol-5-yl)amino]methyl ethyl carbonate |
| A9 | [[4-[(5S)-5-(3,5-dichloro-4-fluoro-phenyl)-5-(trifluoromethyl)-4H-isoxazol-3-yl]-2-methyl- |
| | benzoyl]-(3-ethoxy-4,5-dihydroisoxazol-5-yl)amino]methyl isopropyl carbonate |
| A10 | [[4-[(5S)-5-(3,5-dichloro-4-fluoro-phenyl)-5-(trifluoromethyl)-4H-isoxazol-3-yl]-2-methyl- |
| | benzoyl]-(3-ethoxy-4,5-dihydroisoxazol-5-yl)amino]methyl propanoate |
| A11 | [[4-[(5S)-5-(3,5-dichloro-4-fluoro-phenyl)-5-(trifluoromethyl)-4H-isoxazol-3-yl]-2-methyl- |
| | benzoyl]-(3-ethoxy-4,5-dihydroisoxazol-5-yl)amino]methyl acetate |

or an agrochemically acceptable salt, tautomer or N-oxide thereof.

The term “a compound of formula (I)” or “a compound of the invention” as used herein has the meaning of a compound according to any one of embodiments 1 to 10.

Compounds of formula (I) include at least one chiral centre and may exist as compounds of formula (I*) or compounds of formula (I**):

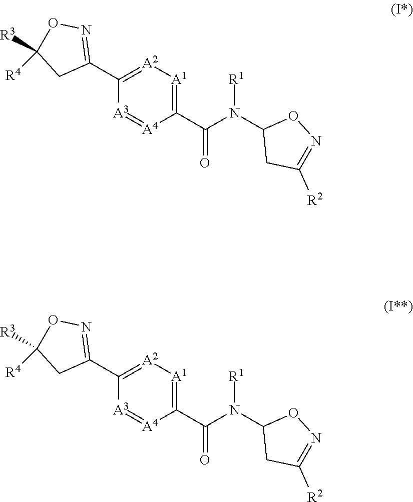

Generally compounds of formula (I**) are more biologically active than compounds of formula (I*).

The invention includes mixtures of compounds (I*) and (I**) in any ratio e.g. in a molar ratio of 1:99 to 99:1, e.g. 10:1 to 1:10, e.g. a substantially 50:50 molar ratio. In an enantiomerically (or epimerically) enriched mixture of formula (I**), the molar proportion of compound (I**) compared to the total amount of both enantiomers (or epimers) is for example greater than 50%, e.g. at least 55, 60, 65, 70, 75, 80, 85, 90, 95, 96, 97, 98, or at least 99%. Likewise, in enantiomerically (or epimerically) enriched mixture of formula (I*), the molar proportion of the compound of formula (I*) compared to the total amount of both enantiomers (or epimers) is for example greater than 50%, e.g. at least 55, 60, 65, 70, 75, 80, 85, 90, 95, 96, 97, 98, or at least 99%. Enantiomerically (or epimerically) enriched mixtures of formula (I**) are preferred.

The compounds of the invention may be made by a variety of methods as shown in Schemes 1 to 9.

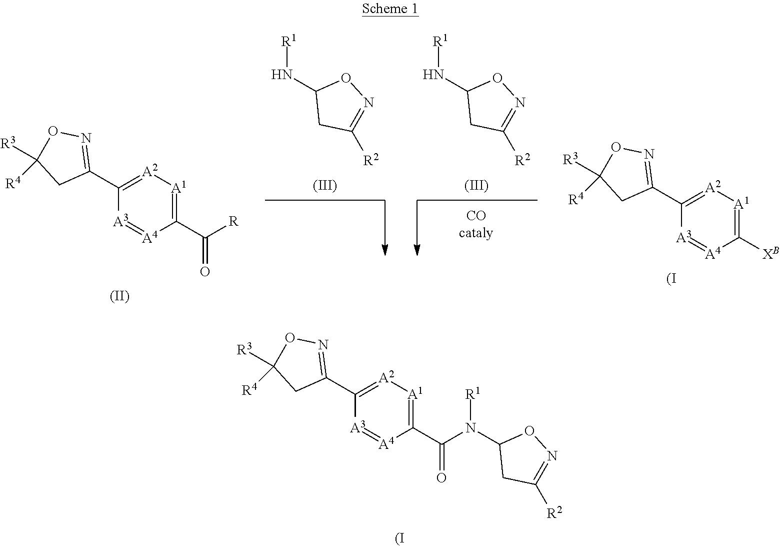

1) Compounds of formula (I) can be prepared by reacting a compound of formula (II) wherein R is OH, C₁-C₆alkoxy or C₁, F or Br, with an amine of formula (III), as shown in Scheme 1. When R is OH such reactions are usually carried out in the presence of a coupling reagent, such as N,N-dicyclohexylcarbodiimide (“DCC”), 1-ethyl-3-(3-dimethylamino-propyl)carbodiimide hydrochloride (“EDO”) or bis(2-oxo-3-oxazolidinyl)phosphonic chloride (“BOP—CI”), in the presence of a base, and optionally in the presence of a nucleophilic catalyst, such as hydroxybenzotriazole (“HOBT”). When R is C₁, such reactions are usually carried out in the presence of a base, and optionally in the presence of a nucleophilic catalyst. Alternatively, it is possible to conduct the reaction in a biphasic system comprising an organic solvent, preferably ethyl acetate, and an aqueous solvent, preferably a solution of sodium hydrogen carbonate. When R is C₁-C₆alkoxy it is sometimes possible to convert the ester directly to the amide by heating the ester and amine together in a thermal process. Suitable bases include pyridine, triethylamine, 4-(dimethylamino)-pyridine (“DMAP”) or diisopropylethylamine (Hunig's base). Preferred solvents are N,N-dimethylacetamide, tetrahydrofuran, dioxane, 1,2-dimethoxyethane, ethyl acetate and toluene. The reaction is carried out at a temperature of from 0° C. to 100° C., preferably from 15° C. to 30° C., in particular at ambient temperature. Amines of formula (III) can be prepared using method 11 described below.

2) Acid halides of formula (II), wherein R is C₁, F or Br, may be made from carboxylic acids of formula (II), wherein R is OH, under standard conditions, as described for example in WO09080250.

3) Carboxylic acids of formula (II), wherein R is OH, may be formed from esters of formula (II), wherein R is C₁-C₆alkoxy as described for example in WO09080250.

4) Compounds of formula (I) can be prepared by reacting a compound of formula (IV) wherein X^{B} is a leaving group, for example a halogen, such as bromo, with carbon monoxide and an amine of formula (III), in the presence of a catalyst, such as palladium(II) acetate or bis-(triphenylphosphine)palladium(II) dichloride, optionally in the presence of a ligand, such as triphenylphosphine, and a base, such as sodium carbonate, pyridine, triethylamine, 4-(dimethylamino)-pyridine (“DMAP”) or diisopropylethylamine (Hunig's base), in a solvent, such as water, N,N-dimethylformamide or tetrahydrofuran. The reaction is carried out at a temperature of from 50° C. to 200° C., preferably from 100° C. to 150° C. The reaction is carried out at a pressure of from 50 to 200 bar, preferably from 100 to 150 bar.

5) Compounds of formula (IV) wherein X^{B} is a leaving group, for example a halogen, such as bromo, can be made by a various of methods, for example as described in WO09080250.

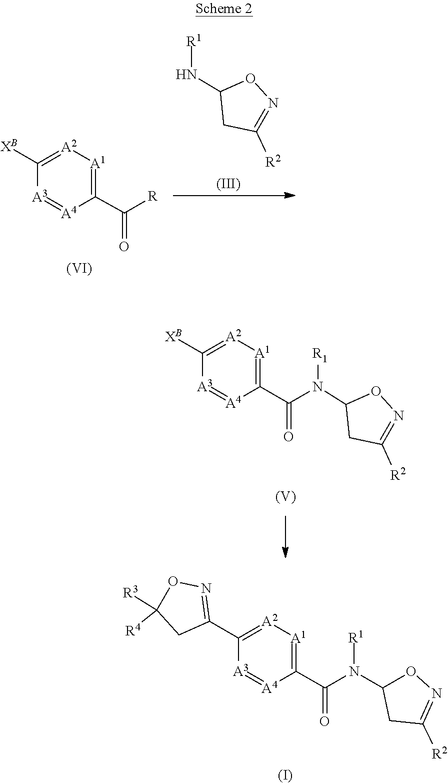

6) Alternatively, compounds of formula (I) can be prepared by various methods from an intermediate of formula (V) as shown in Scheme 2 wherein X^{B} is a leaving group, for example a halogen, such as bromo, or X^{B} is cyano, formyl or acetyl according to similar methods to those described in WO09080250. An intermediate of formula (V) can be prepared for example from an intermediate of formula (VI) as described in the same reference.

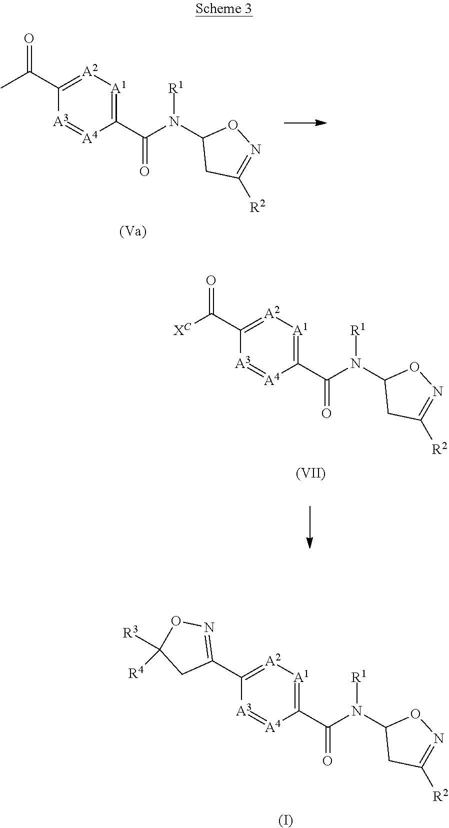

7) Alternatively, compounds of formula (I) can be prepared by various methods from an intermediate of formula (VII) as shown in Scheme 3 wherein X^{C} is CH═C(R³)R⁴, or CH₂C(OH)(R³)R⁴ wherein R³ and R⁴ are as defined for a compound of formula (I) according to similar methods to those described in WO09080250.

8) Compounds of formula (VII) wherein X^{C} is CH═C(R³)R⁴, or CH₂C(OH)(R³)R⁴ can be prepared from a compound of formula (Va) or from a compound of formula (VII) wherein X^{C} is CH₂-halogen using similar methods to those described in WO09080250.

9) Compounds of formula (VII) wherein X^{C} is CH₂-halogen, such as bromo or chloro, can be prepared by reacting a methyl ketone of formula (Va), with a halogenating agent, such as bromine or chlorine, in a solvent, such as acetic acid, at a temperature of from 0° C. to 50° C., preferably from ambient temperature to 40° C.

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylamnnoniunn hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reaction is advantageously carried out in a temperature range from approximately −80° C. to approximately +140° C., preferably from approximately −30° C. to approximately +100° C., in many cases in the range between ambient temperature and approximately +80° C.

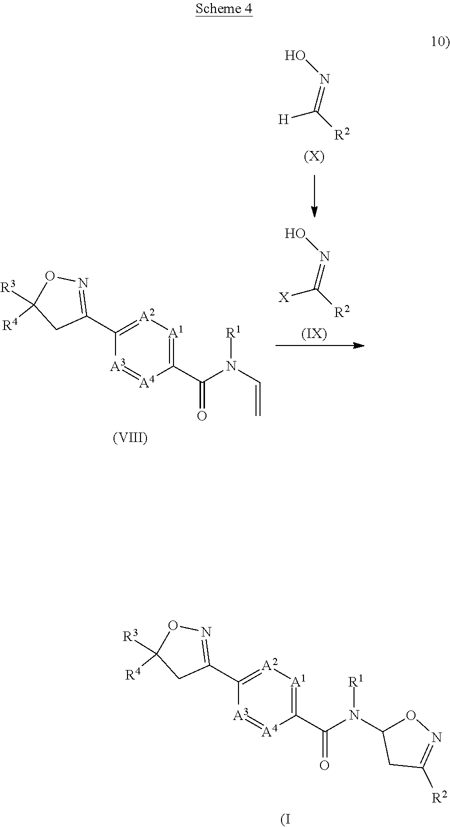

Compounds of formula (I) can be made by reaction of an oxime of formula (X) and a vinyl compound of formula (VIII) in a two step reaction. In the first step, the oxime of formula (X) is reacted with a halogenating agent, for example chlorine, or a succinimide, such as N-chlorosuccinimide (“NCS”), in the presence of a suitable solvent, for example a polar solvent, such as N,N-dimethylformamide, to obtain a compounds of formula (IX) wherein X is a halogen, e.g. chloro. The first step is carried out at a temperature of from 0° C. to 100° C., preferably from 15° C. to 30° C., in particular at ambient temperature.

In the second step, the halo hydroxy imine intermediate of formula (IX), e.g. chloro hydroxy imine, is reacted with the vinyl compound of formula (VIII) in the presence of a base, for example an organic base, such as triethylamine, or an inorganic base, such as sodium hydrogen carbonate, in the presence of a suitable solvent, for example a polar solvent, such as N,N-dimethylformamide or isopropanol or an apolar solvent, such as toluene. It is possible to conduct these two steps separately and optionally to isolate the halo hydroxy imine intermediate or more conveniently to conduct these two steps successively in one reaction vessel without isolation of the intermediate. The second step is carried out at a temperature of from 0° C. to 100° C., preferably from 15° C. to 30° C., in particular at ambient temperature. Vinyl compounds of formula (VIII) are easily prepared using methods known to a person skilled in the art, such as is described in WO2013120940.

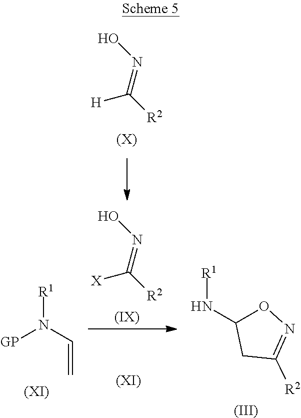

11) Compounds of formula (III) can be made by reaction of an oxime of formula (X) and a vinyl compound of formula (XI) in a two step reaction, such as is described in 10). Vinyl compounds of formula (XI), wherein PG is a protecting group, as described by Greene's Protective Groups in Organic Synthesis, Peter G. M. Wuts (Author), John Wiley & Sons; 5^{th} Edition (23. Dec. 2014), ISBN-10: 1118057481, ISBN-13: 978-1118057483, see chapter 7 especially chapter 7 part C, are easily prepared using methods known to a person skilled in the art, or are commercially available.

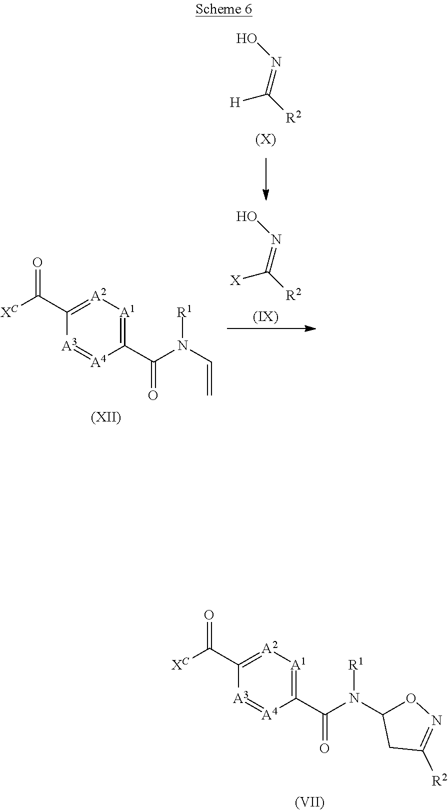

12) Compounds of formula (VII) can be made by reaction of an oxime of formula (X) and a vinyl compound of formula (XII) in a two step reaction, such as is described in 10). Vinyl compounds of formula (XII) are easily prepared using methods known to a person skilled in the art such as is described in WO2013120940.

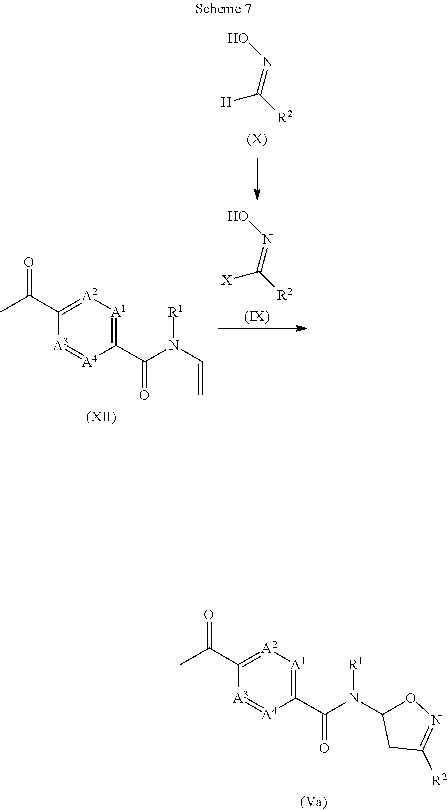

13) Compounds of formula (Va) can be made by reaction of an oxime of formula (X) and a vinyl compound of formula (XIII) in a two-step reaction, such as is described in 10). Vinyl compounds of formula (XIII) are easily prepared using methods known to a person skilled in the art such as is described in WO2013120940.

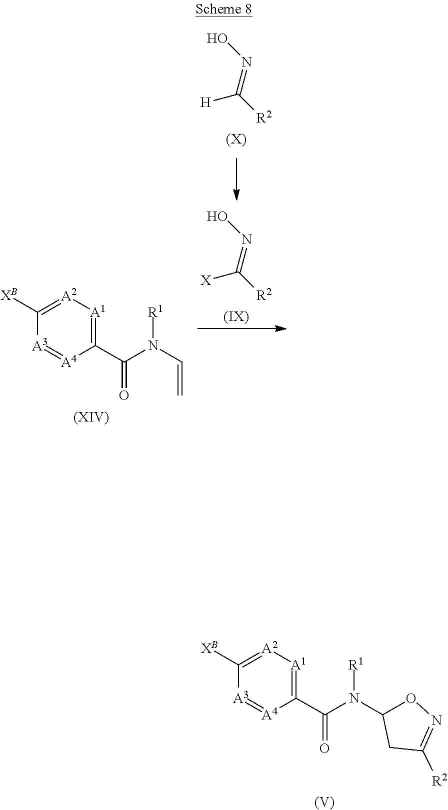

14) Compounds of formula (V) can be made by reaction of an oxime of formula (X) and a vinyl compound of formula (XIV) in a two-step reaction, such as is described in 10). Vinyl compounds of formula (XIV) are easily prepared using methods known to a person skilled in the art such as is described in WO2013120940.

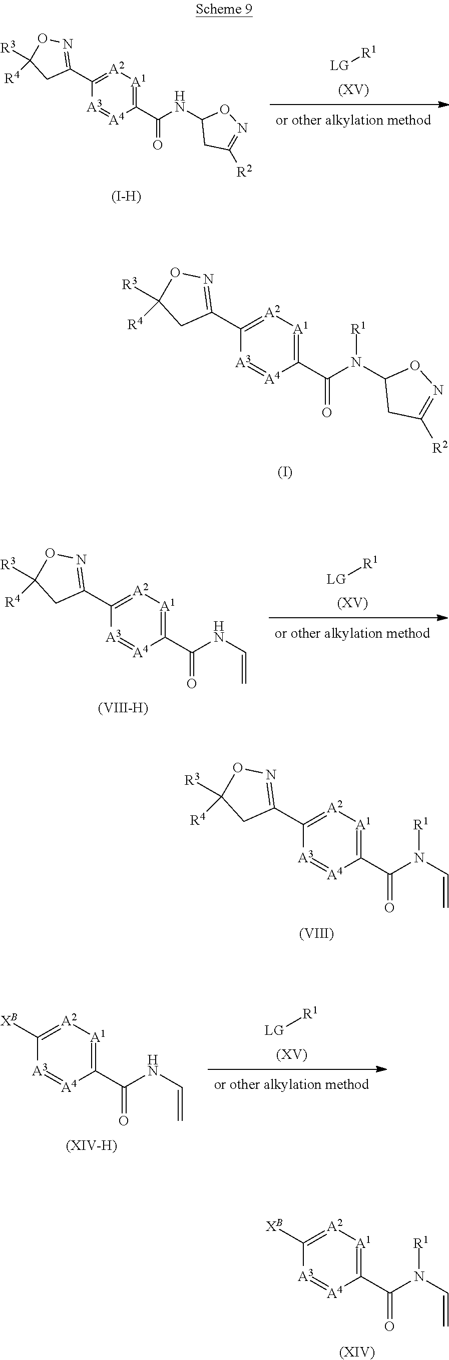

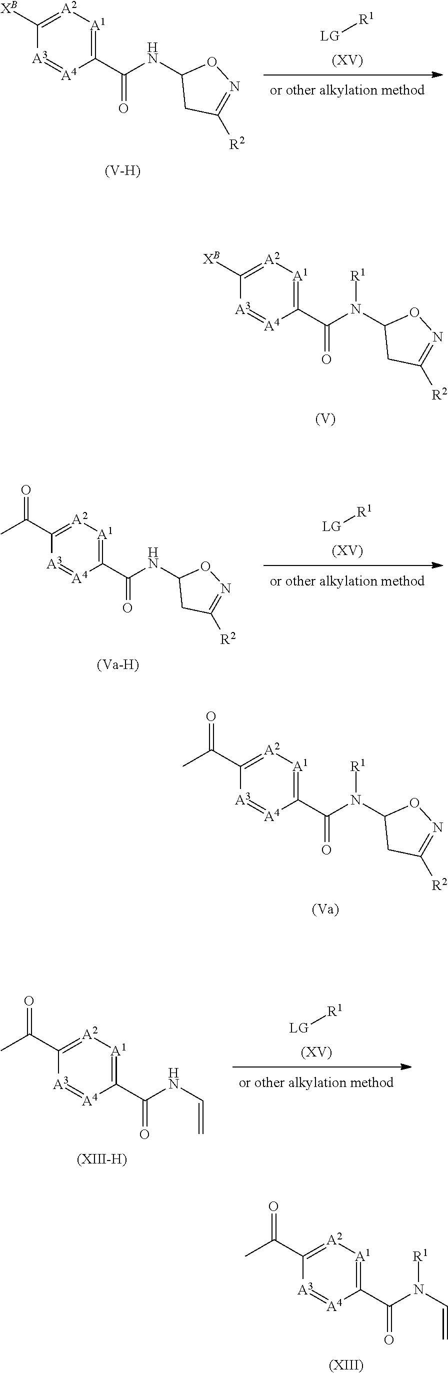

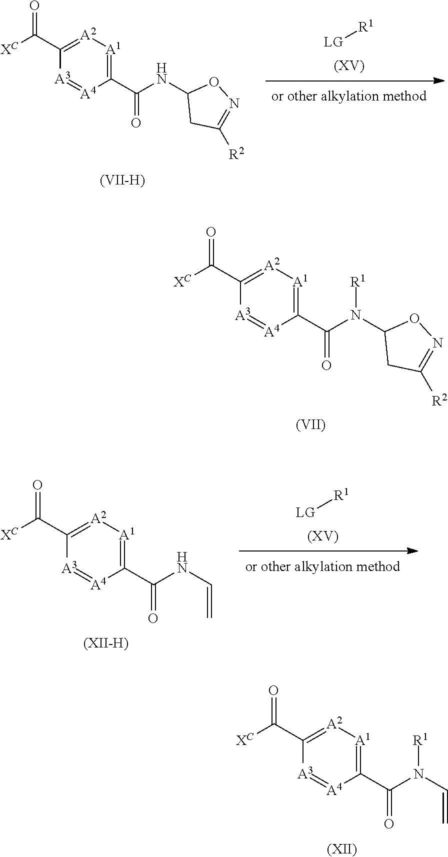

11) Compounds of formula (I) can be prepared by reacting a compound of formula (l-H) with a compound of formula (XV) where LG is a suitable leaving group, for example a halogen, such as bromo or chloro, or an imidazole or an alkylsulfoxy group or a haloalkylsulfoxy group or an arylsulfoxy group, as shown in Scheme 5. Such reactions are usually carried out in the presence of a base, such as sodium hydroxide or sodium hydride, using methods known to a person skilled in the art.

13) Similarly, compounds of formula (VIII), (XIV), (V), (Va), (XIII), (VII) and (XII) can be prepared by reacting a compound of formula (VIII-H), (XIV-H), (V-H), (Va-H), (XIII-H, (VII-H) and (XII-H) respectively with a compound of formula (VIII).

A compound of formula (I) can be converted in a manner known per se into another compound of formula (I) by replacing one or more substituents of the starting compound of formula (I) in the customary manner by (an)other substituent(s) according to the invention.

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reaction is advantageously carried out in a temperature range from approximately −80° C. to approximately +140° C., preferably from approximately −30° C. to approximately +100° C., in many cases in the range between ambient temperature and approximately +80° C.

A compound of formula (I) can be converted in a manner known per se into another compound of formula (I) by replacing one or more substituents of the starting compound of formula (I) in the customary manner by (an)other substituent(s) according to the invention. Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

A compound of formula (I) can be converted in a manner known per se into another compound of formula (I) by replacing one or more substituents of the starting compound of formula (I) in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula (I) can be prepared in a manner known per se. Thus, for example, acid addition salts of compounds of formula (I) are obtained by treatment with a suitable acid or a suitable ion exchanger reagent and salts with bases are obtained by treatment with a suitable base or with a suitable ion exchanger reagent.

Salts of compounds of formula (I) can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula (I) can be converted in a manner known per se into other salts of compounds of formula (I), acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula (I), which have salt-forming properties can be obtained in free form or in the form of salts.

The compounds of formula (I) and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula (I), in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula (I) with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO200015615.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula (I) and, where appropriate, the tautomers thereof, in each case in free form or in salt form, can, if appropriate, also be obtained in the form of hydrates and/or include other solvents, for example those which may have been used for the crystallization of compounds which are present in solid form.

The present invention also provides intermediates useful for the preparation of compounds of formula (I). Certain intermediates are novel and as such form a further aspect of the invention. One group of novel intermediates are compounds of formula (Int-I)

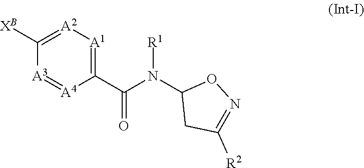

wherein A¹, A², A³, A⁴, R¹ and R² are as defined for a compound of formula (I) and X^{B} is a halogen, such as bromo, or X^{B} is cyano, formyl, CH═N—OH or acetyl; or a salt or N-oxide thereof. The preferences for A¹, A², A³, A⁴, R¹ and R² are the same as the preferences set out for the corresponding substituents of a compound of formula (I).

Another group of novel intermediates are compounds of formula (Int-II)

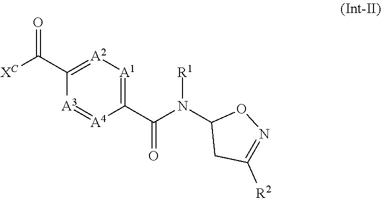

wherein A¹, A², A³, A⁴, R¹ and R² are as defined for a compound of formula (I); X^{C} is Ch₂-halogen, wherein halogen is preferably, bromo or chloro, CH═C(R³)R⁴ or CH₂C(OH)(R³)R⁴ wherein R³ and R⁴ are as defined for a compound of formula (I); or a salt or N-oxide thereof. The preferences for A¹, A², A³, A⁴, R¹ and R² are the same as the preferences set out for the corresponding substituents of a compound of formula (I).

Another group of novel intermediates are compounds of formula (Int-III)

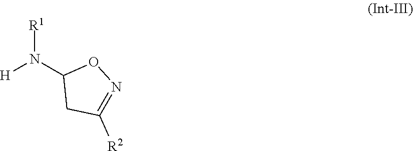

wherein R¹ and R² are as defined for a compound of formula (I); or a salt or N-oxide thereof. The preferences for R¹ and R² are the same as the preferences set out for the corresponding substituents of a compound of formula (I).

Another group of novel intermediates are compounds of formula (Int-IV)

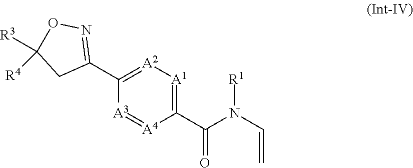

wherein A¹, A², A³, A⁴, R¹, R³ and R⁴ are as defined for a compound of formula (I) or a salt or N-oxide thereof. The preferences for A¹, A², A³, A⁴, R¹, R³ and R⁴ are the same as the preferences set out for the corresponding substituents of a compound of formula (I).

The compounds of formula (I) according to the invention are preventively and/or curatively valuable active ingredients in the field of pest control, even at low rates of application, which have a very favorable biocidal spectrum and are well tolerated by warm-blooded species, fish and plants. The pests which may be combated and controlled by the use of the compounds of the invention include those pests associated with agriculture (which term includes the growing of crops for food and fiber products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies). The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate, a good activity corresponding to a destruction rate (mortality) of at least 50 to 60%.

Examples of the abovementioned animal pests are:
from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,
Haematopinus spp., Linognathus spp., Pediculus spp., Pemphigus spp. and Phylloxera spp.;
from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens;

Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Ouadraspidiotus spp., Ouesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., Cephus spp., Diprion spp., Diprionidae, Gilpinia polytoma, Hoplocampa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate
from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,
Damalinea spp. and Trichodectes spp.;
from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,
Liposcelis spp.;
from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

The active ingredients according to the invention may be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperfiorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime). Coreopsis spp., Crassula coccinea, Cuphea ignea. Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Beilis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata). Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum. A. cepa, A. oschaninii, A. Porrum, A. ascalonicum, A. fistulosum), Anthriscus cerefolium, Apium graveolus. Asparagus officinalis. Beta vulgarus, Brassica spp. (B. Oleracea, B. Pekinensis, B. rapa), Capsicum annuum, Cicer anetinum, Cichorium endivia, Cichorum spp. (C. intybus, C. endivia), Citrillus lanatus, Cucumis spp. (C. sativus, C. melo), Cucurbita spp. (C. pepo. C. maxima), Cyanara spp. (C. scolymus, C. cardunculus), Daucus carota, Foeniculum vulgare, Hypericum spp., Lactuca sativa, Lycopersicon spp. (L esculentum, L. iycopersicum), Mentha spp., Ocimum basilicum, Petroselinum crispum, Phaseolus spp. (P. vulgaris, P. coccineus), Pisum sativum, Raphanus sativus. Rheum rhaponticum, Rosemarinus spp., Salvia spp., Scorzonera hispanica, Solanum meiongena, Spinacea oieracea, Valerianella spp. (V. locusta, V. eriocarpa) and Vida faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatoes) and Chilo supressalis (preferably in rice).

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatoes) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Umax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g. imazamox, by conventional methods of breeding is Clearfield® summer canola. Examples of crops that have been rendered tolerant to herbicides by genetic engineering methods include e.g. glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include δ-endotoxins, vegetative insecticidal proteins (Vip), insecticidal proteins of bacteria colonising nematodes, and toxins produced by scorpions, arachnids, wasps and fungi.

An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which may be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which may be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO2008151984, WO2003034823, U.S. Pat. No. 5,631,072, WO2005064072, WO2006128870, EP1724392, WO2005113886 or WO2007090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables X and Y:

TABLE X
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE Y
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, |
| | | Serviceberry, Hawthorn |
| | Chrysobothris | Apple, Apricot, Beech, Boxelder, |
| | femorata | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, |
| | | Black oak, Cherrybark oak, |
| | | Water oak, Sycamore |
| | Goes tigrinus | Oak |
| | Neoclytus | Ash, Hickory, Oak, Walnut, |
| | acuminatus | Birch, Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, |
| | | Sassafras, Lilac, Mountain- |
| | | mahogany, Pear, Cherry, Plum, |
| | | Peach, Apple, Elm, Basswood, |
| | | Sweetgum |
| | Neoptychodes | Fig, Alder, Mulberry, Willow, |
| | trilineatus | Netleaf hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honey- |
| | | locust, Dogwood, Eucalyptus, |
| | | Oak, Hackberry, Maple, Fruit |
| | | trees |
| | Saperda calcarata | Poplar |
| | Strophiona nitens | Chestnut, Oak, Hickory, Walnut, |
| | | Beech, Maple |
| Scolytidae | Corthylus | Maple, Oak, Yellow-poplar, |
| | columbianus | Beech, Boxelder, Sycamore, |
| | | Birch, Basswood, Chestnut, Elm |
| | Dendroctonus | Pine |
| | frontalis | |
| | Dryocoetes betulae | Birch, Sweetgum, Wild cherry, |
| | | Beech, Pear |
| | Monarthrum | Oak, Maple, Birch, Chestnut, |
| | fasciatum | Sweetgum, Blackgum, Poplar, |
| | | Hickory, Mimosa, Apple, Peach, |
| | | Pine |
| | Phloeotribus | Peach, Cherry, Plum, Black |
| | liminaris | cherry, Elm, Mulberry, |
| | | Mountain-ash |
| | Pseudopityophthorus | Oak, American beech, Black |
| | pruinosus | cherry, Chickasaw plum, |
| | | Chestnut, Maple, Hickory, |
| | | Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, |
| | | Black Cherry |
| | Synanthedon | Tupelo |
| | rubrofascia | |
| | Synanthedon scitula | Dogwood, Pecan, Hickory, Oak, |
| | | Chestnut, Beech, Birch, Black |
| | | cherry, Elm, Mountain-ash, |
| | | Viburnum, Willow, Apple, |
| | | Loquat, Ninebark, Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa afncana) and leatherjackets (European crane fly, Tipula spp).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:
Of the order Anoplurida: Haematopinus spp., Linognathus spp., Pediculus spp. and Phtirus spp., Solenopotes spp.
Of the order Mallophagida: Trimenopon spp., Menopon spp., Trinoton spp., Bovicola spp., Werneckiella spp., Lepikentron spp., Damalina spp., Trichodectes spp. and Felicola spp.
Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.
Of the order Siphonapterida, for example Pulex spp., Ctenocephalides spp., Xenopsylla spp., Ceratophyllus spp.
Of the order Heteropterida, for example Cimex spp., Triatoma spp., Rhodnius spp., Panstrongylus spp.
Of the order Blattarida, for example Blatta orientalis, Periplaneta americana, Blattelagermanica and Supella spp.
Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.
Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention may be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec, and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes 35 darwiniensis, Zootermopsis nevadensis and Coptotermes formosanus, and bristletails such as Lepisma saccharina.

The compounds according to the invention may be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethyleneglycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they may be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that may be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

The activity of compositions comprising compounds according to the invention can be broadened considerably, and adapted to prevailing circumstances, by including other active substances. The active substances can be of chemical or biological in type, and in the case of biological could be further modified from the biological species derived in nature. Active substances include substances that control, repel or attract pests that damage or harm useful plants in general, but also substances that improve the growth of a useful plant, such as plant growth regulators, and substances that improve the performance of the active substance, such as synergists. Examples are insecticides, acaricides, nematicides, molluscicides, aligicides, virusicides, rodenticide, bactericides, fungicides, chemosterilants, anthelmintics. Examples of a biological active substance include baculovirus, plant extract, and bacteria.

The mixtures of the compounds of formula (I) with other active substances may also have further surprising advantages which can also be described, in a wider sense, as synergistic activity. For example, better tolerance by plants, reduced phytotoxicity, insects can be controlled in their different development stages, or better behaviour relating to production, for example grinding or mixing, storage or use.

Individual active substances can occur in more than one group or class, and at more than one place within a group or class: information about the active substances, their spectrum, sources and classifications can be found from Compendium of Pesticide Common Names (see http://www.alanwood.net/pesticides/index.html) or from the Pesticide Manual created by the British Crop Production Counci (see http://bcpcdata.com/pesticide-manual.html).

Preferred mixtures are indicated below where a compound according to any one of embodiments 1 to 10 is indicated as “I”. In particular, “I” represents a compound according to embodiment 10. Compositions comprising an adjuvant include I+compounds selected from the group of substances consisting of petroleum oils;

Compositions comprising an acaricide include I+1,1-bis(4-chlorophenyl)-2-ethoxyethanol, I+2,4-dichlorophenyl benzenesulfonate, I+2-fluoro-N-methyl-N-1-naphthylacetamide, I+4-chlorophenyl phenyl sulfone, I+abamectin, I+acequinocyl, I+acetoprole, I+acrinathrin, I+aldicarb, I+aldoxycarb, I+alpha-cypermethrin, I+amidithion, I+amidoflumet, I+amidothioate, I+amiton, I+amiton hydrogen oxalate, I+amitraz, I+aramite, I+arsenous oxide, I+AVI 382, I+AZ 60541, I+azinphos-ethyl, I+azinphos-methyl, I+azobenzene, I+azocyclotin, I+azothoate, I+benomyl, I+benoxafos, I+benzoximate, I+benzyl benzoate, I+bifenazate, I+bifenthrin, I+binapacryl, I+brofenvalerate, I+bromocyclen, I+bromophos, I+bromophos-ethyl, I+bromopropylate, I+buprofezin, I+butocarboxim, I+butoxycarboxim, I+butylpyridaben, I+calcium polysulfide, I+camphechlor, I+carbanolate, I+carbaryl, I+carbofuran, I+carbophenothion, I+CGA 50′439, I+chinomethionat, I+chlorbenside, I+chlordimeform, I+chlordimeform hydrochloride, I+chlorfenapyr, I+chlorfenethol, I+chlorfenson, I+chlorfensulfide, I+chlorfenvinphos, I+chlorobenzilate, I+chloromebuform, I+chloromethiuron, I+chloropropylate, I+chlorpyrifos, I+chlorpyrifos-methyl, I+chlorthiophos, I+cinerin I, I+cinerin II, I+cinerins, I+clofentezine, I+closantel, I+coumaphos, I+crotamiton, I+crotoxyphos, I+cufraneb, I+cyanthoate, I+cyflumetofen, I+cyhalothrin, I+cyhexatin, I+cypermethrin, I+DCPM, I+DDT, I+demephion, I+demephion-O, I+demephion-S, I+demeton, I+demeton-methyl, I+demeton-O, I+demeton-O-methyl, I+demeton-S, I+demeton-S-methyl, I+demeton-S-methylsulfon, I+diafenthiuron, I+dialifos, I+diazinon, I+dichlofluanid, I+dichlorvos, I+dicliphos, I+dicofol, I+dicrotophos, I+dienochlor, I+dimefox, I+dimethoate, I+dinactin, I+dinex, I+dinex-diclexine, I+dinobuton, I+dinocap, I+dinocap-4, I+dinocap-6, I+dinocton, I+dinopenton, I+dinosulfon, I+dinoterbon, I+dioxathion, I+diphenyl sulfone, I+disulfiram, I+disulfoton, I+DNOC, I+dofenapyn, I+doramectin, I+endosulfan, I+endothion, I+EPN, I+eprinomectin, I+ethion, I+ethoate-methyl, I+etoxazole, I+etrimfos, I+fenazaflor, I+fenazaquin, I+fenbutatin oxide, I+fenothiocarb, I+fenpropathrin, I+fenpyrad, I+fenpyroximate, I+fenson, I+fentrifanil, I+fenvalerate, I+fipronil, I+fluacrypyrim, I+fluazuron, I+flubenzimine, I+flucycloxuron, I+flucythrinate, I+fluenetil, I+flufenoxuron, I+flumethrin, I+fluorbenside, I+fluvalinate, I+FMC 1137, I+formetanate, I+formetanate hydrochloride, I+formothion, I+formparanate, I+gamma-HCH, I+glyodin, I+halfenprox, I+heptenophos, I+hexadecyl cyclopropanecarboxylate, I+hexythiazox, I+iodomethane, I+isocarbophos, I+isopropyl 0-(methoxyaminothiophosphoryl)salicylate, I+ivermectin, I+jasmolin I, I+jasmolin II, I+jodfenphos, I+lindane, I+lufenuron, I+malathion, I+malonoben, I+mecarbam, I+mephosfolan, I+mesulfen, I+methacrifos, I+methamidophos, I+methidathion, I+methiocarb, I+methomyl, I+methyl bromide, I+metolcarb, I+mevinphos, I+mexacarbate, I+milbemectin, I+milbemycin oxime, I+mipafox, I+monocrotophos, I+morphothion, I+moxidectin, I+naled, I+NC-184, I+NC-512, I+nifluridide, I+nikkomycins, I+nitrilacarb, I+nitrilacarb 1:1 zinc chloride complex, I+NNI-0101, I+NNI-0250, I+omethoate, I+oxamyl, I+oxydeprofos, I+oxydisulfoton, I+pp′-DDT, I+parathion, I+permethrin, I+petroleum oils, I+phenkapton, I+phenthoate, I+phorate, I+phosalone, I+phosfolan, I+phosmet, I+phosphamidon, I+phoxim, I+pirimiphos-methyl, I+polychloroterpenes, I+polynactins, I+proclonol, I+profenofos, I+promacyl, I+propargite, I+propetamphos, I+propoxur, I+prothidathion, I+prothoate, I+pyrethrin I, I+pyrethrin II, I+pyrethrins, I+pyridaben, I+pyridaphenthion, I+pyrimidifen, I+pyrimitate, I+quinalphos, I+quintiofos, I+R-1492, I+RA-17, I+rotenone, I+schradan, I+sebufos, I+selamectin, I+SI-0009, I+sophamide, I+spirodiclofen, I+spiromesifen, I+SSI-121, I+sulfiram, I+sulfluramid, I+sulfotep, I+sulfur, I+SZI-121, I+tau-fluvalinate, I+tebufenpyrad, I+TEPP, I+terbam, I+tetrachlorvinphos, I+tetradifon, I+tetranactin, I+tetrasul, I+thiafenox, I+thiocarboxime, I+thiofanox, I+thiometon, I+thioquinox, I+thuringiensin, I+triamiphos, I+triarathene, I+triazophos, I+triazuron, I+trichlorfon, I+trifenofos, I+trinactin, I+vamidothion, I+vaniliprole and I+YI-5302;

Compositions comprising an anthelmintic include I+abamectin, I+crufomate, I+doramectin, I+emamectin, I+emamectin benzoate, I+eprinomectin, I+ivermectin, I+milbemycin oxime, I+moxidectin, I+piperazine, I+selamectin, I+spinosad and I+thiophanate; Compositions comprising an avicide include I+chloralose, I+endrin, I+fenthion, I+pyridin-4-amine and I+strychnine;

Compositions comprising a biological control agent include I+Adoxophyes orana GV, I+Agrobacterium radiobacter, I+Amblyseius spp., I+Anagrapha falcifera NPV, I+Anagrus atomus, I+Aphelinus abdominalis, I+Aphidius colemani, I+Aphidoletes aphidimyza, I+Autographa californica NPV, I+Bacillus firmus, I+Bacillus sphaericus Neide, I+Bacillus thuringiensis Berliner, I+Bacillus thuringiensis subsp. aizawai, I+Bacillus thuringiensis subsp. israelensis, I+Bacillus thuringiensis subsp. japonensis, I+Bacillus thuringiensis subsp. kurstaki, I+Bacillus thuringiensis subsp. tenebrionis, I+Beauveria bassiana, I+Beauveria brongniartii, I+Chrysoperla carnea, I+Cryptolaemus montrouzieri, I+Cydia pomonella GV, I+Dacnusa sibirica, I+Diglyphus isaea, I+Encarsia formosa, I+Eretmocerus eremicus, I+Helicoverpa zea NPV, I+Heterorhabditis bacteriophora and H. megidis, I+Hippodamia convergens, I+Leptomastix dactylopii, I+Macrolophus caliginosus, I+Mamestra brassicae NPV, I+Metaphycus helvolus, I+Metarhizium anisopliae var. acridum, I+Metarhizium anisopliae var. anisopliae, I+Neodiprion sertifer NPV and N. lecontei NPV, I+Onus spp., I+Paecilomyces fumosoroseus, I+Paecilomyces lilacinus, I+Phytoseiulus persimilis, I+Spodoptera exigua multicapsid nuclear polyhedrosis virus, I+Steinernema bibionis, I+Steinernema carpocapsae, I+Steinernema feltiae, I+Steinernema glaseri, I+Steinernema nobrave, I+Steinernema riobravis, I+Steinernema scapterisci, I+Steinernema spp., I+Trichogramma spp., I+Typhlodromus occidentalis, I+Quillaja saponaria, I+Neem tree based products, I+Granulovirus, and I+Verticillium lecanii;

Compositions comprising a soil sterilant include I+iodomethane and methyl bromide;

Compositions comprising a chemosterilant include I+apholate, I+bisazir, I+busulfan, I+diflubenzuron, I+dimatif, I+hemel, I+hempa, I+metepa, I+methiotepa, I+methyl apholate, I+morzid, I+penfluron, I+tepa, I+thiohempa, I+thiotepa, I+tretamine and I+uredepa;

Compositions comprising an insect pheromone include I+(E)-dec-5-en-1-yl acetate with (E)-dec-5-en-1-ol, I+(E)-tridec-4-en-1-yl acetate, I+(E)-6-methylhept-2-en-4-ol, I+(E,Z)-tetradeca-4,10-dien-1-yl acetate, I+(Z)-dodec-7-en-1-yl acetate, I+(Z)-hexadec-11-enal, I+(Z)-hexadec-11-en-1-yl acetate, I+(Z)-hexadec-13-en-11-yn-1-yl acetate, I+(Z)-icos-13-en-10-one, I+(Z)-tetradec-7-en-1-al, I+(Z)-tetradec-9-en-1-ol, I+(Z)-tetradec-9-en-1-yl acetate, I+(7E,9Z)-dodeca-7,9-dien-1-yl acetate, I+(9Z,11E)-tetradeca-9,11-dien-1-yl acetate, I+(9Z,12E)-tetradeca-9,12-dien-1-yl acetate, I+14-methyloctadec-1-ene, I+4-methylnonan-5-ol with 4-methylnonan-5-one, I+alpha-multistriatin, I+brevicomin, I+codlelure, I+codlemone, I+cuelure, I+disparlure, I+dodec-8-en-1-yl acetate, I+dodec-9-en-1-yl acetate, I+dodeca-8, I+10-dien-1-yl acetate, I+dominicalure, I+ethyl 4-methyloctanoate, I+eugenol, I+frontalin, I+gossyplure, I+grandlure, I+grandlure I, I+grandlure II, I+grandlure III, I+grandlure IV, I+hexalure, I+ipsdienol, I+ipsenol, I+japonilure, I+lineatin, I+litlure, I+looplure, I+medlure, I+megatomoic acid, I+methyl eugenol, I+muscalure, I+octadeca-2,13-dien-1-yl acetate, I+octadeca-3,13-dien-1-yl acetate, I+orfralure, I+oryctalure, I+ostramone, I+siglure, I+sordidin, I+sulcatol, I+tetradec-11-en-1-yl acetate, I+trimedlure, I+trimedlure A, I+trimedlure B₁, I+trimedlure B₂, I+trimedlure C and I+trunc-call; Compositions comprising an insect repellent include I+2-(octylthio)ethanol, I+butopyronoxyl, I+butoxy(polypropylene glycol), I+dibutyl adipate, I+dibutyl phthalate, I+dibutyl succinate, I+diethyltoluamide, I+dimethyl carbate, I+dimethyl phthalate, I+ethyl hexanediol, I+hexamide, I+methoquin-butyl, I+methylneodecanamide, I+oxamate and I+picaridin;

Compositions comprising an insecticide include I+1-dichloro-1-nitroethane, I+1,1-dichloro-2,2-bis(4-ethylphenyl)ethane, I+, I+1,2-dichloropropane, I+1,2-dichloropropane with 1,3-dichloropropene, I+1-bromo-2-chloroethane, I+2,2,2-trichloro-1-(3,4-dichlorophenyl)ethyl acetate, I+2,2-dichlorovinyl 2-ethylsulfinylethyl methyl phosphate, I+2-(1,3-dithiolan-2-yl)phenyl dimethylcarbamate, I+2-(2-butoxyethoxy)ethyl thiocyanate, I+2-(4,5-dimethyl-1,3-dioxolan-2-yl)phenyl methylcarbamate, I+2-(4-chloro-3,5-xylyloxy)ethanol, I+2-chlorovinyl diethyl phosphate, I+2-imidazolidone, I+2-isovalerylindan-1,3-dione, I+2-methyl(prop-2-ynyl)aminophenyl methylcarbamate, I+2-thiocyanatoethyl laurate, I+3-bromo-1-chloroprop-1-ene, I+3-methyl-1-phenylpyrazol-5-yl dimethylcarbamate, I+4-methyl(prop-2-ynyl)amino-3,5-xylyl methylcarbamate, I+5,5-dimethyl-3-oxocyclohex-1-enyl dimethylcarbamate, I+abamectin, I+acephate, I+acetamiprid, I+acethion, I+acetoprole, I+acrinathrin, I+acrylonitrile, I+alanycarb, I+aldicarb, I+aldoxycarb, I+aldrin, I+allethrin, I+allosamidin, I+allyxycarb, I+alpha-cypermethrin, I+alpha-ecdysone, I+aluminium phosphide, I+amidithion, I+amidothioate, I+aminocarb, I+amiton, I+amiton hydrogen oxalate, I+amitraz, I+anabasine, I+athidathion, I+AVI 382, I+AZ 60541, I+azadirachtin, I+azamethiphos, I+azinphos-ethyl, I+azinphos-methyl, I+azothoate, I+Bacillus thuringiensis delta endotoxins, I+barium hexafluorosilicate, I+barium polysulfide, I+barthrin, I+Bayer 22/190, I+Bayer 22408, I+bendiocarb, I+benfuracarb, I+bensultap, I+beta-cyfluthrin, I+beta-cypermethrin, I+bifenthrin, I+bioallethrin, I+bioallethrin S-cyclopentenyl isomer, I+bioethanomethrin, I+biopermethrin, I+bioresmethrin, I+bis(2-chloroethyl) ether, I+bistrifluron, I+borax, I+brofenvalerate, I+bromfenvinfos, I+bromocyclen, I+bromo-DDT, I+bromophos, I+bromophos-ethyl, I+bufencarb, I+buprofezin, I+butacarb, I+butathiofos, I+butocarboxim, I+butonate, I+butoxycarboxim, I+butylpyridaben, I+cadusafos, I+calcium arsenate, I+calcium cyanide, I+calcium polysulfide, I+camphechlor, I+carbanolate, I+carbaryl, I+carbofuran, I+carbon disulfide, I+carbon tetrachloride, I+carbophenothion, I+carbosulfan, I+cartap, I+cartap hydrochloride, I+cevadine, I+chlorbicyclen, I+chlordane, I+chlordecone, I+chlordimeform, I+chlordimeform hydrochloride, I+chlorethoxyfos, I+chlorfenapyr, I+chlorfenvinphos, I+chlorfluazuron, I+chlormephos, I+chloroform, I+chloropicrin, I+chlorphoxim, I+chlorprazophos, I+chlorpyrifos, I+chlorpyrifos-methyl, I+chlorthiophos, I+chromafenozide, I+cinerin I, I+cinerin II, I+cinerins, I+cis-resmethrin, I+cismethrin, I+clocythrin, I+cloethocarb, I+closantel, I+clothianidin, I+copper acetoarsenite, I+copper arsenate, I+copper oleate, I+coumaphos, I+coumithoate, I+crotamiton, I+crotoxyphos, I+crufomate, I+cryolite, I+CS 708, I+cyanofenphos, I+cyanophos, I+cyanthoate, I+cyclethrin, I+cycloprothrin, I+cyfluthrin, I+cyhalothrin, I+cypermethrin, I+cyphenothrin, I+cyromazine, I+cythioate, I+d-limonene, I+d-tetramethrin, I+DAEP, I+dazomet, I+DDT, I+decarbofuran, I+deltamethrin, I+demephion, I+demephion-O, I+demephion-S, I+demeton, I+demeton-methyl, I+demeton-O, I+demeton-O-methyl, I+demeton-S, I+demeton-S-methyl, I+demeton-S-methylsulphon, I+diafenthiuron, I+dialifos, I+diamidafos, I+diazinon, I+dicapthon, I+dichlofenthion, I+dichlorvos, I+dicliphos, I+dicresyl, I+dicrotophos, I+dicyclanil, I+dieldrin, I+diethyl 5-methylpyrazol-3-yl phosphate, I+diflubenzuron, I+dilor, I+dimefluthrin, I+dimefox, I+dimetan, I+dimethoate, I+dimethrin, I+dimethylvinphos, I+dimetilan, I+dinex, I+dinex-diclexine, I+dinoprop, I+dinosam, I+dinoseb, I+dinotefuran, I+diofenolan, I+dioxabenzofos, I+dioxacarb, I+dioxathion, I+disulfoton, I+dithicrofos, I+DNOC, I+doramectin, I+DSP, I+ecdysterone, I+EI 1642, I+emamectin, I+emamectin benzoate, I+EMPC, I+empenthrin, I+endosulfan, I+endothion, I+endrin, I+EPBP, I+EPN, I+epofenonane, I+eprinomectin, I+esfenvalerate, I+etaphos, I+ethiofencarb, I+ethion, I+ethiprole, I+ethoate-methyl, I+ethoprophos, I+ethyl formate, I+ethyl-DDD, I+ethylene dibromide, I+ethylene dichloride, I+ethylene oxide, I+etofenprox, I+etrimfos, I+EXD, I+famphur, I+fenamiphos, I+fenazaflor, I+fenchlorphos, I+fenethacarb, I+fenfluthrin, I+fenitrothion, I+fenobucarb, I+fenoxacrim, I+fenoxycarb, I+fenpirithrin, I+fenpropathrin, I+fenpyrad, I+fensulfothion, I+fenthion, I+fenthion-ethyl, I+fenvalerate, I+fipronil, I+flonicamid, I+flubendiamide, I+flucofuron, I+flucycloxuron, I+flucythrinate, I+fluenetil, I+flufenerim, I+flufenoxuron, I+flufenprox, I+flumethrin, I+fluvalinate, I+FMC 1137, I+fonofos, I+formetanate, I+formetanate hydrochloride, I+formothion, I+formparanate, I+fosmethilan, I+fospirate, I+fosthiazate, I+fosthietan, I+furathiocarb, I+furethrin, I+gamma-cyhalothrin, I+gamma-HCH, I+guazatine, I+guazatine acetates, I+GY-81, I+halfenprox, I+halofenozide, I+HCH, I+HEOD, I+heptachlor, I+heptenophos, I+heterophos, I+hexaflumuron, I+HHDN, I+hydramethylnon, I+hydrogen cyanide, I+hydroprene, I+hyquincarb, I+imidacloprid, I+imiprothrin, I+indoxacarb, I+iodomethane, I+IPSP, I+isazofos, I+isobenzan, I+isocarbophos, I+isodrin, I+isofenphos, I+isolane, I+isoprocarb, I+isopropyl 0-(methoxyaminothiophosphoryl)salicyiate, I+isoprothiolane, I+isothioate, I+isoxathion, I+ivermectin, I+jasmolin I, I+jasmolin II, I+jodfenphos, I+juvenile hormone I, I+juvenile hormone II, I+juvenile hormone III, I+kelevan, I+kinoprene, I+lambda-cyhalothrin, I+lead arsenate, I+lepimectin, I+leptophos, I+lindane, I+lirimfos, I+lufenuron, I+lythidathion, I+m-cumenyl methylcarbamate, I+magnesium phosphide, I+malathion, I+malonoben, I+mazidox, I+mecarbam, I+mecarphon, I+menazon, I+mephosfolan, I+mercurous chloride, I+mesulfenfos, I+metaflumizone, I+metam, I+metam-potassium, I+metam-sodium, I+methacrifos, I+methamidophos, I+methanesulfonyl fluoride, I+methidathion, I+methiocarb, I+methocrotophos, I+methomyl, I+methoprene, I+methoquin-butyl, I+methothrin, I+methoxychlor, I+methoxyfenozide, I+methyl bromide, I+methyl isothiocyanate, I+methylchloroform, I+methylene chloride, I+metofluthrin, I+metolcarb, I+metoxadiazone, I+mevinphos, I+mexacarbate, I+milbemectin, I+milbemycin oxime, I+mipafox, I+mirex, I+monocrotophos, I+morphothion, I+moxidectin, I+naftalofos, I+naled, I+naphthalene, I+NC-170, I+NC-184, I+nicotine, I+nicotine sulfate, I+nifluridide, I+nitenpyram, I+nithiazine, I+nitrilacarb, I+nitrilacarb 1:1 zinc chloride complex, I+NNI-0101, I+NNI-0250, I+nornicotine, I+novaluron, I+noviflumuron, I+O-5-dichloro-4-iodophenyl O-ethyl ethylphosphonothioate, I+O,O-diethyl O-4-methyl-2-oxo-2H-chromen-7-yl phosphorothioate, I+O,O-diethyl O-6-methyl-2-propylpyrimidin-4-yl phosphorothioate, I+O,O,O′,O′-tetrapropyi dithiopyrophosphate, I+oleic acid, I+omethoate, I+oxamyl, I+oxydemeton-methyl, I+oxydeprofos, I+oxydisulfoton, I+pp′-DDT, I+para-dichlorobenzene, I+parathion, I+parathion-methyl, I+penfluron, I+pentachlorophenol, I+pentachlorophenyl laurate, I+permethrin, I+petroleum oils, I+PH 60-38, I+phenkapton, I+phenothrin, I+phenthoate, I+phorate+TX, I+phosalone, I+phosfolan, I+phosmet, I+phosnichlor, I+phosphamidon, I+phosphine, I+phoxim, I+phoxim-methyl, I+pirimetaphos, I+pirimicarb, I+pirimiphos-ethyl, I+pirimiphos-methyl, I+polychlorodicyclopentadiene isomers, I+polychloroterpenes, I+potassium arsenite, I+potassium thiocyanate, I+prallethrin, I+precocene I, I+precocene II, I+precocene III, I+primidophos, I+profenofos, I+profluthrin, I+promacyl, I+promecarb, I+propaphos, I+propetamphos, I+propoxur, I+prothidathion, I+prothiofos, I+prothoate, I+protrifenbute, I+pymetrozine, I+pyraclofos, I+pyrazophos, I+pyresmethrin, I+pyrethrin I, I+pyrethrin II, I+pyrethrins, I+pyridaben, I+pyridalyl, I+pyridaphenthion, I+pyrimidifen, I+pyrimitate, I+pyriproxyfen, I+quassia, I+quinalphos, I+quinalphos-methyl, I+quinothion, I+quintiofos, I+R-1492, I+rafoxanide, I+resmethrin, I+rotenone, I+RU 15525, I+RU 25475, I+ryania, I+ryanodine, I+sabadilla, I+schradan, I+sebufos, I+selamectin, I+SI-0009, I+SI-0205, I+SI-0404, I+SI-0405, I+silafluofen, I+SN 72129, I+sodium arsenite, I+sodium cyanide, I+sodium fluoride, I+sodium hexafluorosilicate, I+sodium pentachlorophenoxide, I+sodium selenate, I+sodium thiocyanate, I+sophamide, I+spinosad, I+spiromesifen, I+spirotetrmat, I+sulcofuron, I+sulcofuron-sodium, I+sulfluramid, I+sulfotep, I+sulfuryl fluoride, I+sulprofos, I+tar oils, I+tau-fluvalinate, I+tazimcarb, I+TDE, I+tebufenozide, I+tebufenpyrad, I+tebupirimfos, I+teflubenzuron, I+tefluthrin, I+temephos, I+TEPP, I+terallethrin, I+terbam, I+terbufos, I+tetrachloroethane, I+tetrachlorvinphos, I+tetramethrin, I+theta-cypermethrin, I+thiacloprid, I+thiafenox, I+thiamethoxam, I+thicrofos, I+thiocarboxime, I+thiocyclam, I+thiocyclam hydrogen oxalate, I+thiodicarb, I+thiofanox, I+thiometon, I+thionazin, I+thiosultap, I+thiosultap-sodium, I+thuringiensin, I+tolfenpyrad, I+tralomethrin, I+transfluthrin, I+transpermethrin, I+triamiphos, I+triazamate, I+triazophos, I+triazuron, I+trichlorfon, I+trichlormetaphos-3, I+trichloronat, I+trifenofos, I+triflumuron, I+trimethacarb, I+triprene, I+vamidothion, I+vaniliprole, I+veratridine, I+veratrine, I+XMC, I+xylylcarb, I+YI-5302, I+zeta-cypermethrin, I+zetamethrin, I+zinc phosphide, I+zolaprofos and ZXI 8901, I+cyantraniliprole, I+chlorantraniliprole, I+cyenopyrafen, I+cyflumetofen, I+pyrifluquinazon, I+spinetoram, I+spiropidion, I+sulfoxaflor, I+flufiprole, I+meperfluthrin, I+tetramethylfluthrin, I+triflumezopyrim, I+Afidopyropen, I+flupyrimin, I+Broflanilide, I+Cyhalodiamide, I+Fluxametamide, I+Momfluorothrin, I+kappa-bifenthrin, I+kappa-tefluthrin, I+Dichloromezotiaz, I+Epsilon-metofluthrin, I+Epsilon-momfluorothrin, I+Cyclaniliprole, I+Tetrachloraniliprole, I benzpyrimoxan;

Compositions comprising a molluscicide include I+bis(tributyltin) oxide, I+bromoacetamide, I+calcium arsenate, I+cloethocarb, I+copper acetoarsenite, I+copper sulfate, I+fentin, I+ferric phosphate, I+metaldehyde, I+methiocarb, I+niclosamide, I+niclosamide-olamine, I+pentachlorophenol, I+sodium pentachlorophenoxide, I+tazimcarb, I+thiodicarb, I+tributyltin oxide, I+trifenmorph, I+trimethacarb, I+triphenyltin acetate and triphenyltin hydroxide, I+pyriprole;

Compositions comprising a nematicide include l+AKD-3088, I+1,2-dibromo-3-chloropropane, I+1,2-dichloropropane, I+1,2-dichloropropane with 1,3-dichloropropene, I+1,3-dichloropropene, I+3,4-dichlorotetrahydrothiophene 1,1-dioxide, I+3-(4-chlorophenyl)-5-methylrhodanine, I+5-methyl-6-thioxo-1,3,5-thiadiazinan-3-ylacetic acid, I+6-isopentenylaminopurine, I+abamectin, I+acetoprole, I+alanycarb, I+aldicarb, I+aldoxycarb, I+AZ 60541, I+benclothiaz, I+benomyl, I+butylpyridaben, I+cadusafos, I+carbofuran, I+carbon disulfide, I+carbosulfan, I+chloropicrin, I+chlorpyrifos, I+cloethocarb, I+cytokinins, I+dazomet, I+DBCP, I+DCIP, I+diamidafos, I+dichlofenthion, I+dicliphos, I+dimethoate, I+doramectin, I+emamectin, I+emamectin benzoate, I+eprinomectin, I+ethoprophos, I+ethylene dibromide, I+fenamiphos, I+fenpyrad, I+fensulfothion, I+fosthiazate, I+fosthietan, I+furfural, I+GY-81, I+heterophos, I+iodomethane, I+isamidofos, I+isazofos, I+ivermectin, I+kinetin, I+mecarphon, I+metam, I+metam-potassium, I+metam-sodium, I+methyl bromide, I+methyl isothiocyanate, I+milbemycin oxime, I+moxidectin, I+Myrothecium verrucaria composition, I+NC-184, I+oxamyl, I+phorate, I+phosphamidon, I+phosphocarb, I+sebufos, I+selamectin, I+spinosad, I+terbam, I+terbufos, I+tetrachlorothiophene, I+thiafenox, I+thionazin, I+triazophos, I+triazuron, I+xylenols, I+YI-5302 and zeatin, I+fluensulfone, I+fluopyram, I+Iprodione, I+Tioxazafen, I+Fluazaindolizine; Compositions comprising a synergist include I+2-(2-butoxyethoxy)ethyl piperonylate, I+5-(1,3-benzodioxol-5-yl)-3-hexylcyclohex-2-enone, I+farnesol with nerolidol, I+MB-599, I+MGK 264, I+piperonyl butoxide, I+piprotal, I+propyl isomer, I+S421, I+sesamex, I+sesasmolin and I+sulfoxide;

Compositions comprising an animal repellent include I+anthraquinone, I+chloralose, I+copper naphthenate, I+copper oxychloride, I+diazinon, I+dicyclopentadiene, I+guazatine, I+guazatine acetates, I+methiocarb, I+pyridin-4-amine, I+thiram, I+trimethacarb, I+zinc naphthenate and I+ziram;

Further compositions include I+Brofluthrinate, I+Cycloxaprid, I+Diflovidazine, I+Flometoquin, I+Fluhexafon, I+Guadipyr, I+Plutella xylostella Granulosis virus, I+Cydia pomonella Granulosis virus, I+Harpin, I+Imicyafos, I+Heliothis virescens Nucleopolyhedrovirus, I+Heliothis punctigera Nucleopolyhedrovirus, I+Helicoverpa armigera Nucleopolyhedrovirus, I+Helicoverpa zea Nucleopolyhedrovirus, I+Spodoptera frugiperda Nucleopolyhedrovirus, I+Plutella xylostella Nucleopolyhedrovirus, I+Pasteuria nishizawae, I+p-cymene, I+Pyflubumide, I+Pyrafluprole, I+pyrethrum, I+QRD 420, I+QRD 452, I+QRD 460, I+Terpenoid blends, I+Terpenoids, I+Tetraniliprole, and I+α-terpinene;

Composition also include mixtures of compound of formula (I) and an active substance referenced by a code, such as I+code AE 1887196 (BSC-BX60309), I+code NNI-0745 GR, I+code IKI-3106, I+code JT-L001, I+code ZNQ-08056, I+code IPPA152201, I+code HNPC-A9908 (CAS: [660411-21-2]), I+code HNPC-A2005 (CAS: [860028-12-2]), I+code JS118, I+code ZJ0967, I+code ZJ2242, I+code JS7119 (CAS: [929545-74-4]), I+code SN-1172, I+code HNPC-A9835, I+code HNPC-A9955, I+code HNPC-A3061, I+code Chuanhua 89-1, I+code IPP-10, I+codeZJ3265, I+code JS9117, I+code ZJ3757, I+code ZJ4042, I+code ZJ4014, I+code ITM-121, I+code DPX-RAB55 (DKI-2301), I+code NA-89, I+code MIE-1209, I+code MCI-8007, I+code BCS-CL73507, I+code S-1871, I+code DPX-RDS63, and I+code AKD-1193.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds according to any one of embodiments 1 to 10, in particular according to embodiment 10, and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above may be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula (I) selected from Tables 1 to 48 and Table A and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) selected from Tables 1 to 48 and Table A and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula (I). The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

The invention further relates to a pesticidal composition, which comprises at least one compound of formula (I) according to this invention or at least one compound of formula (Ib) or, where appropriate, a tautomer thereof, in each case in free form or in agrochemically utilizable salt form, as active ingredient.

The invention further relates to a method for controlling pests, which comprises applying a composition according to the invention to the pests or their environment preferably with the exception of a method for treatment of the human or animal body by surgery or therapy and diagnostic methods practised on the human or animal body.

The invention further relates to a method for the protection of plant propagation material from the attack by pests, which comprises treating the propagation material or the site, where the propagation material is planted, with a composition comprising a compound according to this invention or with a compound according to this invention. The invention further relates to a plant propagation material treated with the pesticidal composition comprising a compound according to this invention or with a compound according to this invention.

The Examples which follow serve to illustrate the invention. The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm or 0.8 ppm.

### PREPARATORY EXAMPLES

“Mp” means melting point in ° C. Free radicals represent methyl groups. ¹H NMR measurements were recorded on a Brucker 400 MHz spectrometer, chemical shifts are given in ppm relevant to a TMS standard. Spectra measured in deuterated solvents as indicated.

The following LC-MS methods were used to characterize the compounds:

### Example 1: Preparation of [[4-[(5S)-5-(3,5-dichloro-4-fluoro-phenyl)-5-(trifluoromethyl)-4H-isoxazol-3-yl]-2-methyl-benzoyl]-(3-ethoxy-4,5-dihydroisoxazol-5-yl)amino]methyl ethyl carbonate (Compound A8)

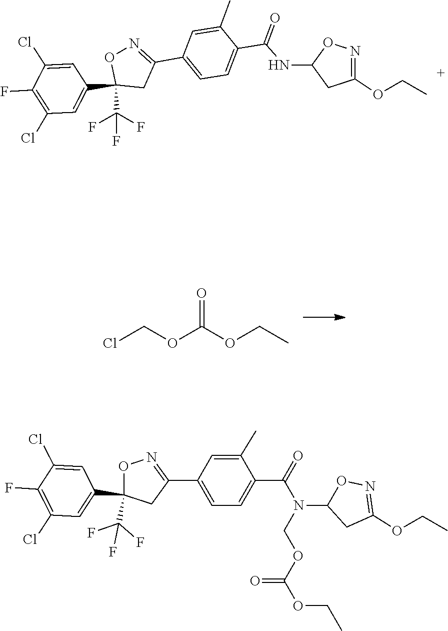

Under a nitrogen atmosphere, a stirred mixture of 4-[(5S)-5-(3,5-dichloro-4-fluoro-phenyl)-5-(trifluoromethyl)-4H-isoxazol-3-yl]-N-(3-ethoxy-4,5-dihydroisoxazol-5-yl)-2-methyl-benzamide (200 mg) in N,N-dimethylformamide (1.82 mL) was cooled to 0° C. After 5 min, sodium hydride (0.022 g, 60% suspension in oil) was added and the reaction mixture was allowed to warm up and to stir at ambient temperature. After 30 min, chloromethyl ethyl carbonate (0.104 g) was added and the reaction mixture was stirred for 2.5 hours. The reaction mixture was carefully quenched with water and diluted with ethyl acetate. The aqueous layer was extracted twice with ethyl acetate and the combined organic layers were washed once with brine, dried over magnesium sulfate, filtered and concentrated under reduced pressure to give a crude product which was purified by Combiflash chromatography (eluent: gradient Cyclohexane/Ethyl acetate 1:0 to 70:30), to give 82 mg of the desired product A8 as a mixture of rotamers. ¹H NMR (400 MHz, CDCl₃) δ ppm 7.47-7.65 (m, 4H) 7.32 (br s, 1H) 6.09-6.78 (m, 1H) 4.86-5.98 (m, 2H) 4.04-4.33 (m, 5H) 3.69 (br d, 1H) 2.99-3.60 (m, 2H) 2.36 (s, 3H) 1.22-1.42 (m, 6H). ¹⁹F NMR (377 MHz, CDCl₃) δ ppm −79.43 and −79.48 (m, 3 F)-113.54 (s, 1 F).

The other examples were prepared in a similar manner using the appropriate intermediates.

| | | | | ¹⁹F NMR δ |
|---|---|---|---|---|
| | | | | ppm (377 |
| | | | | MHz in |
| | | | | CDCl₃, |
| Com- | | | ¹H NMR δ ppm (400 | unless |
| pound | | | MHz in CDCl₃, unless | indicated |
| number | IUPAC name | Structure | indicated otherwise) | otherwise) |
| A1 | N-(cyclopropanecarbonyl)-4- [(5S)-5-(3,5-dichloro-4- fluoro-phenyl)-5- (trifluoromethyl)-4H- isoxazol-3-yl]-N-(3-ethoxy- 4,5-dihydroisoxazol-5-yl)-2- methyl-benzamide | 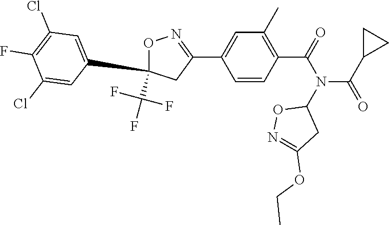 [SMILES: CCOC1=NOC(N(C(=O)c2ccc(C3=NO[C@@](c4cc(Cl)c(F)c(Cl)c4)(C(F)(F)F)C3)cc2C)C(=O)C2CC2)C1] | 7.59 (d, 3 H) 7.49- 7.54 (m, 2 H) 6.57- 6.64 (m, 1 H) 4.16- 4.30 (m, 2 H) 4.09 (d, 1 H) 3.69 (d, 1 H) 3.24- 3.39 (m, 2 H) 2.52 (s, 3 H) 1.48-1.54 (m, 1 H) 1.37 (t, 3 H) 0.83-1.10 (m, 2 H) 0.57-0.75 (m, 2 H) | −79.53 and −79.54 (m, 3 F) −113.39 (s, 1 F) |
| A2 | [[4-[(5S)-5-(3,5-dichloro-4- fluoro-phenyl)-5- (trifluoromethyl)-4H- isoxazol-3-yl]-2-methyl- benzoyl]-(3-ethoxy-4,5- dihydroisoxazol-5- yl)amino]methyl 2- methylpropanoate | 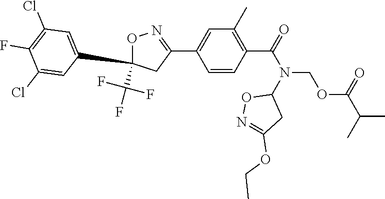 [SMILES: CCOC1=NOC(N(COC(=O)C(C)C)C(=O)c2ccc(C3=NO[C@@](c4cc(Cl)c(F)c(Cl)c4)(C(F)(F)F)C3)cc2C)C1] | 7.24-7.65 (m, 5 H) 6.74-4.84 (m, 3 H) 4.13-4.26 (m, 2 H) 4.09 (dd, 1 H) 3.63- 3.76 (m, 1 H) 2.91- 3.58 (m, 2 H) 2.41- 2.70 (m, 1 H) 2.36 (s, 3 H) 1.30-1.40 (m, 3 H) 1.04-1.28 (m, 6 H) | −79.38 and −79.58 (m, 3 F) −113.56 (s, 1 F) |
| A3 | [[4-[(5S)-5-(3,5-dichloro-4- fluoro-phenyl)-5- (trifluoromethyl)-4H- isoxazol-3-yl]-2-methyl- benzoyl]-(3-ethoxy-4,5- dihydroisoxazol-5- yl)amino]methyl 2,2- dimethylpropanoate | 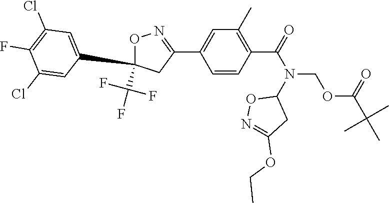 [SMILES: CCOC1=NOC(N(COC(=O)C(C)(C)C)C(=O)c2ccc(C3=NO[C@@](c4cc(Cl)c(F)c(Cl)c4)(C(F)(F)F)C3)cc2C)C1] | 7.59 (d, 2 H) 7.52 (m, 2 H) 7.20-7.35 (m, 1 H) 4.95-6.85 (m, 3 H) 4.14-4.25 (m, 2 H) 4.09 (dd, 1 H) 3.69 (d, 1 H) 3.53 (br dd, 1 H) 3.01 (br s, 1 H) 2.35 (s, 3 H) 1.35 (t, 3 H) 1.12- 1.27 (m, 9 H) | −79.37 and −79.48 (m, 3 F) −113.58 (s, 1 F) |
| A4 | methyl N-[[[4-[(5S)-5-(3,5- dichloro-4-fluoro-phenyl)-5- (trifluoromethyl)-4H- isoxazol-3-yl]-2-methyl- benzoyl]-(3-ethoxy-4,5- dihydroisoxazol-5- yl)amino]methyl]-N-methyl- carbamate | 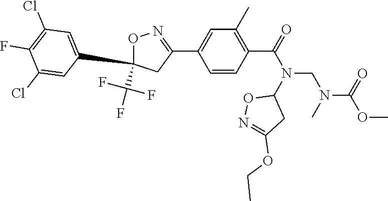 [SMILES: CCOC1=NOC(N(CN(C)C(=O)OC)C(=O)c2ccc(C3=NO[C@@](c4cc(Cl)c(F)c(Cl)c4)(C(F)(F)F)C3)cc2C)C1] | ¹H NMR (400 MHz, acetone) δ ppm 7.81 (d, 2 H) 7.65-7.73 (m, 2 H) 7.30-7.45 (m, 1 H) 5.17-6.07 (m, 2 H) 4.38-4.46 (m, 1 H) 4.27 (dd, 1 H) 4.02- 4.16 (m, 2 H) 3.67 (br m, 3 H) 2.70-3.49 (m, 6 H) 2.27-2.42 (m, 3 H) 1.28 (br t, 3 H) | ¹⁹F NMR (377 MHz, acetone) δ ppm −80.37 and −80.40 (m, 3 F) −116.32 (s, 1 F) |
| A5 | [[4-[(5S)-5-(3,5-dichloro-4- fluoro-phenyl)-5- (trifluoromethyl)-4H- isoxazol-3-yl]-2-methyl- benzoyl]-(3-ethoxy-4,5- dihydroisoxazol-5- yl)amino]methyl N,N- dimethylcarbamate | 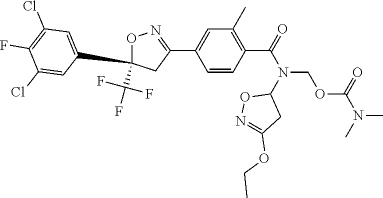 [SMILES: CCOC1=NOC(N(COC(=O)N(C)C)C(=O)c2ccc(C3=NO[C@@](c4cc(Cl)c(F)c(Cl)c4)(C(F)(F)F)C3)cc2C)C1] | 7.28-7.63 (m, 5 H) 6.57-6.81 and 5.06- 6.04 (m, 3 H) 4.21 (q, 2 H) 4.09 (dd, 1 H) 3.69 (dd, 1 H) 3.12-3.58 (m, 1 H) 2.82-3.07 (m, 7 H) 2.36 (s, 3 H) 1.35 (t, 3 H) | −79.34 and −79.41 (m, 3 F) −113.58 (s, 1 F) |
| A6 | [[4-[(5S)-5-(3,5-dichloro-4- fluoro-phenyl)-5- (trifluoromethyl)-4H- isoxazol-3-yl]-2-methyl- benzoyl]-(3-ethoxy-4,5- dihydroisoxazol-5- yl)amino]methyl methyl carbonate | 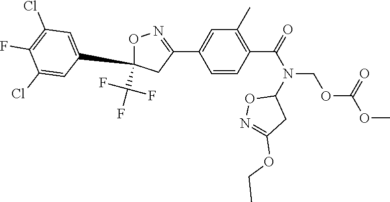 [SMILES: CCOC1=NOC(N(COC(=O)OC)C(=O)c2ccc(C3=NO[C@@](c4cc(Cl)c(F)c(Cl)c4)(C(F)(F)F)C3)cc2C)C1] | 7.30-7.70 (m, 5 H) 6.50-6.79 and 5.05- 5.84 (m, 3 H) 4.21 (q, 2 H) 4.03-4.13 (m, 1 H) 3.62-3.91 (m, 4 H) 2.95-3.60 (m, 2 H) 2.36 (s, 3 H) 1.36 (t, 3 H) | −79.42 and −79.47 (m, 3 F) −113.51 (s, 1 F) |
| A7 | [[4-[(5S)-5-(3,5-dichloro-4- fluoro-phenyl)-5- (trifluoromethyl)-4H- isoxazol-3-yl]-2-methyl- benzoyl]-(3-ethoxy-4,5- dihydroisoxazol-5- yl)amino]methyl cyclopropanecarboxylate | 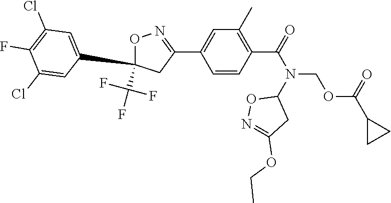 [SMILES: CCOC1=NOC(N(COC(=O)C2CC2)C(=O)c2ccc(C3=NO[C@@](c4cc(Cl)c(F)c(Cl)c4)(C(F)(F)F)C3)cc2C)C1] | 7.29-7.67 (m, 5 H) 6.65-6.83 and 5.02- 6.11 (m, 3 H) 4.15- 4.27 (m, 2 H) 4.09 (dd, 1 H) 3.69 (d, 1 H) 2.94- 3.59 (m, 2 H) 2.36 (s, 3 H) 1.55-1.48 (m, 1 H) 1.37 (br t, 3 H) 1.09- 0.83 (m, 4 H) | −79.38 and −79.55 (m, 3 F) −113.55 (s, 1 F) |
| A8 | [[4-[(5S)-5-(3,5-dichloro-4- fluoro-phenyl)-5- (trifluoromethyl)-4H- isoxazol-3-yl]-2-methyl- benzoyl]-(3-ethoxy-4,5- dihydroisoxazol-5- yl)amino]methyl ethyl carbonate | 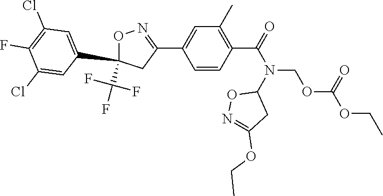 [SMILES: CCOC(=O)OCN(C(=O)c1ccc(C2=NO[C@@](c3cc(Cl)c(F)c(Cl)c3)(C(F)(F)F)C2)cc1C)C1CC(OCC)=NO1] | 7.47-7.65 (m, 4 H) 7.32 (br s, 1 H) 6.09- 6.78 (m, 1 H) 4.86- 5.98 (m, 2 H) 4.04- 4.33 (m, 5 H) 3.69 (br d, 1 H) 2.99-3.60 (m, 2 H) 2.36 (s, 3 H) 1.22- 1.42 (m, 6 H) | −79.43 and −79.48 (m, 3 F) −113.54 (s, 1 F) |
| A9 | [[4-[(5S)-5-(3,5-dichloro-4- fluoro-phenyl)-5- (trifluoromethyl)-4H- isoxazol-3-yl]-2-methyl- benzoyl]-(3-ethoxy-4,5- dihydroisoxazol-5- yl)amino]methyl isopropyl carbonate | 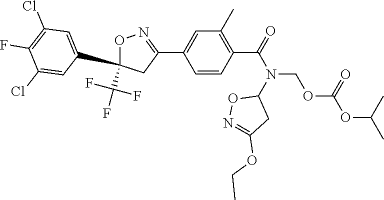 [SMILES: CCOC1=NOC(N(COC(=O)OC(C)C)C(=O)c2ccc(C3=NO[C@@](c4cc(Cl)c(F)c(Cl)c4)(C(F)(F)F)C3)cc2C)C1] | 7.48-7.64 (m, 4 H) 7.28-7.40 (m, 1 H) 5.53-6.85 (m, 1 H) 4.62-5.45 (m, 3 H) 4.04-4.27 (m, 3 H) 3.69 (br d, 1 H) 2.91- 3.57 (m, 2 H) 2.35 (s, 3 H) 1.21-1.44 (m, 9 H) | −79.44 and −79.48 (m, 3 F) −113.54 (s, 1 F) |
| A10 | [[4-[(5S)-5-(3,5-dichloro-4- fluoro-phenyl)-5- (trifluoromethyl)-4H-isoxazol-3- yl]-2-methyl-benzoyl]-(3-ethoxy- 4,5-dihydroisoxazol-5- yl)amino]methyl propanoate | 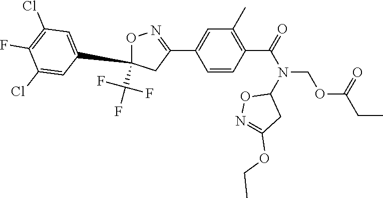 [SMILES: CCOC1=NOC(N(COC(=O)CC)C(=O)c2ccc(C3=NO[C@@](c4cc(Cl)c(F)c(Cl)c4)(C(F)(F)F)C3)cc2C)C1] | 7.46-7.67 (m, 4 H) 7.25- 7.32 (m, 1 H) 6.73 (br s) and 4.94-6.19 (m, 3 H) 4.15-4.26 (m, 2 H) 4.05- 4.15 (m, 1 H) 3.69 (br d, 1 H) 3.34-3.60 (m) and 2.93-3.17 (m, 2 H) 2.16- 2.48 (m, 5 H) 1.36 (br t, 3 H) 1.05-1.22 (m, 3 H) | −79.46 (m, 3 F) −113.55 (s, 1 F) |
| A11 | [[4-[(5S)-5-(3,5-dichloro-4- fluoro-phenyl)-5- (trifluoromethyl)-4H-isoxazol-3- yl]-2-methyl-benzoyl]-(3-ethoxy- 4,5-dihydroisoxazol-5- yl)amino]methyl acetate | 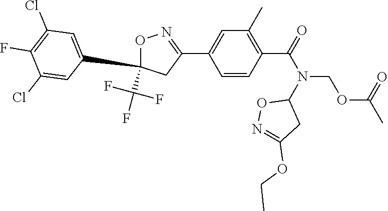 [SMILES: CCOC1=NOC(N(COC(C)=O)C(=O)c2ccc(C3=NO[C@@](c4cc(Cl)c(F)c(Cl)c4)(C(F)(F)F)C3)cc2C)C1] | 7.48-7.66 (m, 4 H) 7.25- 7.32 (m, 1 H) 6.72 (br s) and 4.94-6.13 (m, 3 H) 4.21 (br d, 2 H) 4.09 (dd, 1 H) 3.69 (d, 1 H) 3.35- 3.60 (m) and 2.91-3.16 (m, 2 H) 2.36 (s, 3 H) 1.91- 2.23 (m, 3 H) 1.37 (br t, 3 H) | −79.45 (m, 3 F) −113.54 (s, 1 F) |

### BIOLOGICAL EXAMPLES

These Examples illustrate the pesticidal/insecticidal properties of compounds of formula (I).

Tests were performed as follows:

### Example B1

Diabrotica balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm: A1, A2, A3, A4, A5

### Example B2

Euschistus heros(Neotropical Brown Stink Bug) Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm: A1, A2, A3, A4, A5, A6, A7

### Example B3

Plutella xylostella (Diamond Back Moth)

24-well microtiter plates with artificial diet were treated with aqueous test solutions prepared from 10′000 ppm DMSO stock solutions by pipetting. After drying, Plutella eggs were pipetted through a plastic stencil onto a gel blotting paper and the plate was closed with it. The samples were assessed for mortality and growth inhibition in comparison to untreated samples 8 days after infestation.

The following compounds gave an effect of at least 80% in at least one of the two categories (mortality or growth inhibition) at an application rate of 200 ppm: A4, A5, A6, A7

### Example B4

Plutella xylostella (Diamond Back Moth)

24-well microtiter plates with artificial diet were treated with aqueous test solutions prepared from 10′000 ppm DMSO stock solutions by pipetting. After drying, the plates were infested with L2 larvae (10 to 15 per well). The samples were assessed for mortality and growth inhibition in comparison to untreated samples 5 days after infestation.

The following compounds gave an effect of at least 80% in at least one of the two categories (mortality or growth inhibition) at an application rate of 200 ppm: A1, A2, A3

### Example B5

Myzus persicae (Green Peach Aphid): Feeding/Contact Activity

Sunflower leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm: A1, A2, A3, A4

### Example B6

Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm: A1, A2, A3, A4, A5, A6, A7

### Example B7

Tetranychus urticae (Two-Spotted Spider Mite): Feeding/Contact Activity

Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm: A1, A2, A3, A4, A5

### Example B8

Thrips tabaci (Onion Thrips) Feeding/Contact Activity

Sunflower leaf discs were placed on agar in 24-well microtiter plates and sprayed with aqueous test solutions prepared from 10′000 ppm DMSO stock solutions. After drying the leaf discs were infested with a thrips population of mixed ages. The samples were assessed for mortality 6 days after infestation.

The following compounds resulted in at least 80% mortality at an application rate of 200 ppm: A1, A2, A3, A4, A5

1. A compound of formula (I), 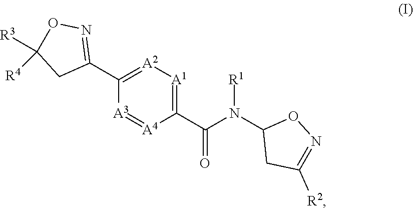
wherein
A¹, A², A³ and A⁴ are, independently of one another, C—H, C—R⁵ or N;
R¹ is selected from —(C₀-C₄alkyl)-C(═O)—C₃-C₆cycloalkyl, —(C₀-C₄alkyl)-O—C(═O)—C₃-C₆cycloalkyl, —(C₀-C₄alkyl)-O—C₃-C₆cycloalkyl, —R^{1a}OR^{1a}OR^{1b}, —R^{1a}OC(═O)R^{1b}, —R^{1a}OC(═O)OR^{1b}, —R^{1a}N(R^{1c})C(═O)OR^{1b}, —R^{1a}OC(═O)N(R^{1b})(R^{1c}), —R^{1a}C(═O)N(R^{1b})(R^{1c}) and —S—C(═O)OR^{1b};
R^{1a} is —(CR^{1d}R^{1e})ₙ—;
R^{1b} and R^{1c} are independently selected from H and C₁-C₄alkyl, wherein each alkyl group is unsubstituted or substituted with one to three halogen atoms or with a cyano group;
R^{1d} and R^{1e} are independently selected from H and C₁-C₄alkyl;
n is selected from 1, 2, 3 and 4;
R² is hydrogen, halogen, cyano, C₁-C₈alkyl or C₁-C₈alkyl substituted by one to three R^{6a}, C₁-C₈haloalkyl or C₁-C₈haloalkyl substituted by one to three R^{6a}, C₁-C₈alkoxy or C₁-C₈alkoxy substituted by one to three R^{6a}, C₃-C₈cycloalkyl or C₃-C₈cycloalkyl substituted by one to three R^{6b}, C₃-C₈cycloalkyl where one carbon atom is replaced by O, S, S(O) or SO₂, or C₃-C₈cycloalkyl-C₁-C₈alkyl, C₃-C₈cycloalkyl-C₁-C₈alkyl where one carbon atom in the cycloalkyl group is replaced by O, S, S(O) or SO₂, or C₃-C₈cycloalkyl-C₁-C₈haloalkyl, C₂-C₈alkenyl or C₂-C₈alkenyl substituted by one to three R^{6a}, C₂-C₈haloalkenyl or C₂-C₈haloalkenyl substituted by one to three R^{6a}, C₂-C₈alkynyl, C₂-C₈haloalkynyl, phenyl, phenyl substituted by one to three R⁷, phenyl-C₁-C₄alkyl, phenyl-C₁-C₄alkyl wherein the phenyl moiety is substituted by one to three R⁷, 5-6 membered heteroaryl, 5-6 membered heteroaryl substituted by one to three R⁷, 5-6 membered heteroaryl-C₁-C₄alkyl or 5-6 membered heteroaryl-C₁-C₄alkyl wherein the heteroaryl moiety is substituted by one to three R⁷, —NH(R⁸), —N(R⁸)(R⁹), —OR¹⁰, —SR¹⁰, —S(O)R¹⁰, —S(O)₂R¹⁰, COR¹⁰, COOR¹⁰;
R³ is C₁-C₈haloalkyl;
R⁴ is phenyl or phenyl substituted by one to three R^{6b} or pyridine or pyridine substituted by one to three R^{6b};
R⁵ is independently halogen, cyano, nitro, C₁-C₈alkyl, C₃-C₈cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy, or C₁-C₈alkoxycarbonyl-, or two R⁵ on adjacent carbon atoms together form a —CH═CH—CH═CH— bridge or a —N═CH—CH═CH— bridge;
each R^{6a} is independently halogen, cyano, nitro, amino, hydroxy, oxo, C₁-C₈alkylamino, hydroxyimino, C₁-C₈alkyloxyimino, di-C₁-C₈alkylamino, C₁-C₈alkoxy, acetyloxy, formyloxy, C₁-C₈haloalkoxy, C₁-C₄alkylthio or tri-(C₁-C₄alkyl)silyl;
each R^{6b} is independently halogen, cyano, nitro, C₁-C₈alkyl, C₁-C₈haloalkyl, amino, C₁-C₈alkylamino, di-C₁-C₈alkylamino, hydroxyl, C₁-C₄alkylthio, C₁-C₈alkoxy or C₁-C₈haloalkoxy;
R⁷ is independently halogen, cyano, nitro, C₁-C₈alkyl, C₁-C₈haloalkyl, C₁-C₈alkoxy, or C₁-C₈haloalkoxy;
R⁸ and R⁹ are independently hydrogen, cyano, C₁-C₈alkyl or C₁-C₈alkyl substituted by one to three R^{6a}, C₁-C₈alkoxy, C₁-C₈haloalkoxy, C₁-C₈haloalkoxy substituted by one to three R^{6a}, C₁-C₈alkoxy substituted by one to three R^{6a}, C₁-C₈haloalkyl or C₁-C₈haloalkyl substituted by one to three R^{6a}, C₃-C₈cycloalkyl or C₃-C₈cycloalkyl substituted by one to three R^{6b}, C₃-C₈cycloalkyl where one carbon atom is replaced by O, S, S(O) or SO₂, or C₃-C₈cycloalkyl-C₁-C₈alkyl, C₃-C₈cycloalkyl-C₁-C₈alkyl where one carbon atom in the cycloalkyl group is replaced by O, S, S(O) or SO₂, or C₃-C₈cycloalkyl-C₁-C₈haloalkyl, C₂-C₈alkenyl or C₂-C₈alkenyl substituted by one to three R^{6a}, C₂-C₈haloalkenyl or C₂-C₈haloalkenyl substituted by one to three R^{6a}, C₂-C₈alkynyl, C₂-C₈haloalkynyl, phenyl, phenyl substituted by one to three R⁷, phenyl-C₁-C₄alkyl, phenyl-C₁-C₄alkyl wherein the phenyl moiety is substituted by one to three R⁷, 5-6 membered heteroaryl, 5-6 membered heteroaryl substituted by one to three R⁷, 5-6 membered heteroaryl-C₁-C₄alkyl or 5-6 membered heteroaryl-C₁-C₄alkyl wherein the heteroaryl moiety is substituted by one to three R⁷, or R⁸ and R⁹ together with the nitrogen atom can be linked through a C₃-C₈alkylene chain, a C₃-C₈alkylene chain substituted by one to three R^{6b} or a C₃-C₈alkylene chain, where one carbon atom is replaced by O, S, S(O) or SO₂;
R¹⁰ is hydrogen, cyano, C₁-C₈alkyl or C₁-C₈alkyl substituted by one to three R^{6a}, C₁-C₈haloalkyl or C₁-C₈haloalkyl substituted by one to three R^{6a}, C₃-C₈cycloalkyl or C₃-C₈cycloalkyl substituted by one to three R^{6b}, C₃-C₈cycloalkyl where one carbon atom is replaced by O, S, S(O) or SO₂, or C₃-C₈cycloalkyl-C₁-C₈alkyl, C₃-C₈cycloalkyl-C₁-C₈alkyl where one carbon atom in the cycloalkyl group is replaced by O, S, S(O) or SO₂, or C₃-C₈cycloalkyl-C₁-C₈haloalkyl, C₂-C₈alkenyl or C₂-C₈alkenyl substituted by one to three R^{6a}, C₂-C₈haloalkenyl or C₂-C₈haloalkenyl substituted by one to three R^{6a}, C₂-C₈alkynyl, C₂-C₈haloalkynyl, phenyl, phenyl substituted by one to three R⁷, phenyl-C₁-C₄alkyl, phenyl-C₁-C₄alkyl wherein the phenyl moiety is substituted by one to three R⁷, 5-6 membered heteroaryl, 5-6 membered heteroaryl substituted by one to three R⁷, 5-6 membered heteroaryl-C₁-C₄alkyl or 5-6 membered heteroaryl-C₁-C₄alky 1 wherein the heteroaryl moiety is substituted by one to three R⁷;
or an agrochemically acceptable salt, tautomer or N-oxide thereof.

2. The compound of formula (I) according to claim 1, wherein A¹ is C—R⁵; A² is C—H; A³ is C—H; and A⁴ is C—H, wherein R⁵ is halogen, cyano, nitro, C₁-C₈alkyl, C₃-C₈cycloalkyl, C₁-C₈haloalkyl, or C₂-C₈alkenyl.

3. The compound of formula (I) according to claim 1, wherein R² is halogen, C₁-C₄alkyl, C₃-C₅cycloalkyl, C₁-C₄haloalkyl, C₁-C₄alkoxy, C₁-C₄haloalkoxy or di-C₁-C₄alkylamino, C₁-C₄alkylthio, C₁-C₄alkyloxycarbonyl.

4. The compound of formula (I) according to claim 1, wherein R² is C₁-C₄alkoxy.

5. The compound of formula (I) according to claim 1, wherein
R¹ is selected from —(C₀-C₄alkyl)-C(═O)—C₃-C₆cycloalkyl, —(C₀-C₄alkyl)-O—C(═O)—C₃-C₆cycloalkyl, —R^{1a}OC(═O)R^{1b}, —R^{1a}OC(═O)OR^{1b}, —R^{1a}N(R^{1c})C(═O)OR^{1b} and —R^{1a}OC(═O)N(R^{1b})(R^{1c}).

6. The compound of formula (I) according to claim 1, wherein
R³ is C₁-C₄haloalkyl.

7. The compound of formula (I) according to claim 1, wherein
R⁴ is phenyl or phenyl substituted by one to three R^{6b}
R^{6b} independently is halogen, cyano, nitro, C₁-C₄alkyl, C₁-C₄haloalkyl, C₁-C₄alkoxy, or C₁-C₄haloalkoxy; more preferably bromo, chloro, fluoro, cyano, nitro, methyl, ethyl, trifluoromethyl, methoxy, difluoromethoxy and trifluoromethoxy

8. The compound of formula (I) according to claim 1 represented by the compound of formula (Ia) 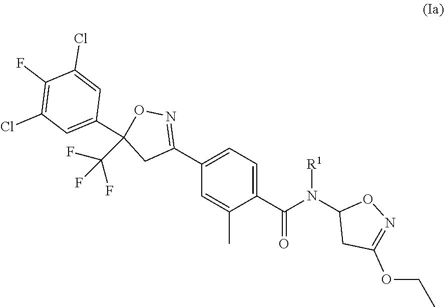
wherein
R¹ is selected from —(C₀-C₄alkyl)-C(═O)—C₃-C₆cycloalkyl, —(C₀-C₄alkyl)-O—C(═O)—C₃-C₆cycloalkyl, —(C₀-C₄alkyl)-O—C₃-C₆cycloalkyl, —R^{1a}OR^{1a}OR^{1b}, —R^{1a}OC(═O)R^{1b}, —R^{1a}OC(═O)OR^{1b}, —R^{1a}N(R^{1c})C(═O)OR^{1b}, —R^{1a}OC(═O)N(R^{1b})(R^{1c}), —R^{1a}C(═O)N(R^{1b})(R^{1c}) and —S—C(═O)OR^{1b};
R^{1a} is —(CR^{1d}R^{1e})ₙ—;
R^{1b} and R^{1c} are independently selected from H and C₁-C₄alkyl, wherein each alkyl group is unsubstituted or substituted with one to three halogen atoms or with a cyano group;
R^{1d} and R^{1e} are independently selected from H and C₁-C₄alkyl;
n is selected from 1, 2, 3 and 4.

9. The compound of claim 8 wherein R¹ is selected from —(C₀-C₄alkyl)-C(═O)—C₃-C₆cycloalkyl, —(C₀-C₄alkyl)-O—C(═O)—C₃-C₆cycloalkyl, —R^{1a}OC(═O)R^{1b}, —R^{1a}OC(═O)OR^{1b}, —R^{1a}N(R^{1c})C(═O)OR^{1b} and —R^{1a}OC(═O)N(R^{1b})(R^{1c}).

10. A compound of formula (Int-I) 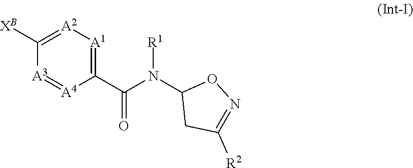
wherein A¹, A², A³, A⁴, R¹ and R² are as defined in claim 1 and X^{B} is a halogen, such as bromo, or X^{B} is cyano, formyl, CH═N—OH or acetyl; or a salt or N-oxide thereof.

11. A compound of formula (Int-II) 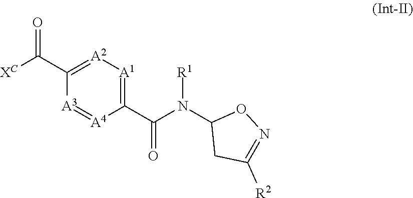
wherein A¹, A², A³, A⁴, R¹ and R² are as defined in claim 1 and X^{C} is CH₂-halogen, CH═C(R³)R⁴ or CH₂C(OH)(R³)R⁴ wherein R³ and R⁴ are as defined for a compound of formula (I); or a salt or N-oxide thereof.

12. A compound of formula (Int-III) 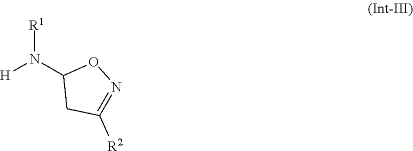
wherein R¹ and R² are as defined in claim 1; or a salt or N-oxide thereof;
or
a compound of formula (Int-IV) 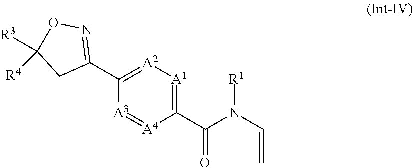
wherein A¹, A², A³, A⁴, R¹, R³ and R⁴ are as defined in claim 1, or a salt or N-oxide thereof.

13. The compound of claim 12, wherein the compound is the compound of formula (Int-IV).

14. A pesticidal composition, which comprises at least one compound of formula (I) according to claim 1 or, where appropriate, a tautomer thereof, in each case in free form or in agrochemically utilizable salt form, as active ingredient.

15. A method for controlling pests, which comprises applying a composition according to claim 14 to the pests or their environment with the exception of a method for treatment of the human or animal body by surgery or therapy and diagnostic methods practised on the human or animal body.

16. The compound of claim 12, wherein the compound is the compound of formula (Int-III).
